(19) United States
(12) Patent Application Publication
(10) Pub. No.: US 2024/0270912 A1
(43) Pub. Date: Aug. 15, 2024

(54) Water-Soluble Films and Methods of Use

(71) Applicant: Nohbo, Inc., Palm Bay, FL (US)

(72) Inventors: Benjamin Gabriel STERN, Indiatlantic, FL (US); Pablo Enrique FERNANDEZ, Rockledge, FL (US)

(21) Appl. No.: 18/442,438

(22) Filed: Feb. 15, 2024

Related U.S. Application Data
(63) Continuation of application No. PCT/US22/74986, filed on Aug. 15, 2022.
(60) Provisional application No. 63/233,446, filed on Aug. 16, 2021.

(51) Int. Cl. C08J 5/18; A61K 8/37; C08L 29/04

(52) U.S. Cl. CPC C08J 5/18; A61K 8/375; C08L 29/04; A61K 2800/87; C08J 2329/04

(57) ABSTRACT

Disclosed herein are shelf-stable water-soluble films containing a hydrophilic polymer blend, and methods of making the same. Also disclosed herein are hygiene product pods comprising a water-soluble envelope and a hygiene product encapsulated therein. Also disclosed herein are methods of making a using a hygiene product pod as described herein.

### CROSS-REFERENCE

This application is a continuation application of PCT/US2022/074986, filed Aug. 15, 2022, which claims the benefit of priority under 35 USC § 119(e) to U.S. Provisional Application No. 63/233,446, filed Aug. 16, 2021, which is incorporated by reference herein in its entirety.

### SUMMARY

Disclosed here are shelf-stable water-soluble films. In some embodiments, a shelf-stable water-soluble film as disclosed herein comprises (a) a hydrophilic polymer blend with a polymeric resin weight average molecular weight of from about 20,000 g/mol to about 80,000 g/mol, wherein the hydrophilic polymer blend comprises: (i) a polyoxazoline polymer, (ii) a polysaccharide, or (iii) a polypeptide; and (b) water content of at least about 1 wt %, with respect to a total weight of the shelf-stable water-soluble film; wherein the shelf-stable water-soluble film at a thickness of about 3 mil when contacted with water at 26° C. has a disintegration time of less than about 30 seconds, and wherein the shelf-stable water-soluble film exhibits stability and lack of degradation for at least about 14 days when stored at 38° C. and 80% relative humidity. In some embodiments, the shelf-stable water-soluble film at a thickness of about 3 mil has a shorter disintegration time when contacted with water at 26° C., relative to a disintegration time under the same conditions of an otherwise comparable film that does not comprise the polyoxazoline polymer, the polysaccharide, or the polypeptide. In some embodiments, the hydrophilic polymer blend further comprises a polyvinyl alcohol polymer. In some embodiments, the polyvinyl alcohol polymer is present in a total amount of from about 50% to about 80% by weight, with respect to a total weight of the shelf-stable water-soluble film. In some embodiments, the polyvinyl alcohol polymer has a net degree of hydrolysis of from about 75% to about 90%. In some embodiments, the water content is from about 1 wt % to about 10 wt %, with respect to the total weight of the shelf-stable water-soluble film. In some embodiments, the hydrophilic polymer blend comprises the polypeptide. In some embodiments, the polypeptide is casein, zein, wheat gluten, whey protein, soy protein, collagen or gelatin. In some embodiments, the hydrophilic polymer blend comprises the polysaccharide. In some embodiments, the polysaccharide is selected from the group consisting of hydroxypropyl methyl cellulose, carboxymethyl cellulose, methyl ether cellulose, microcrystalline cellulose, chitosan, pullulan, methoxyl pectin, alginic acid (e.g. sodium alginate, potassium alginate, magnesium alginate), curdlan, ceral beta glucan, gellan, konjac glucomannan, polydextrose, carrageenan, laminarin, mannan, mannitol, porphyrin, xylan, and a salt of any of these. In some embodiments, the hydrophilic polymer blend comprises the polyoxazoline polymer. In some embodiments, the polyoxazoline polymer is poly(2-ethyl-2-oxazoline). In some embodiments, the poly(2-ethyl-2-oxazoline) is present in an amount of from about 1 wt % to about 25 wt %, with respect to a total weight of the shelf-stable water-soluble film. In some embodiments, the shelf-stable water-soluble film further comprises from about 0.5 wt % to about 5 wt % of a small molecular weight polyhydric alcohol, with respect to a total weight of the shelf-stable water-soluble film. In some embodiments, the small molecular weight polyhydric alcohol has a molecular weight of less than 200 g/mol. In some embodiments, the hydrophilic polymer blend further comprises polyvinylpyrrolidone. In some embodiments, the polyvinylpyrrolidone is present in an amount of from about 1 wt % to about 10 wt %, with respect to a total weight of the shelf-stable water-soluble film. In some embodiments, the polyvinylpyrrolidone has a molecular weight of from about 20,000 g/mol to about 400,000 g/mol. In some embodiments, the shelf-stable water-soluble film further comprises a bubble-reducing component that is present in an amount sufficient to reduce formation of bubbles in the shelf-stable water-soluble film, as compared to an otherwise-comparable film that does not comprise the bubble-reducing component, as determined by heating the shelf-stable water-soluble film and the otherwise comparable film and visualizing the shelf-stable water-soluble film and the otherwise comparable film after cooling. In some embodiments, the bubble-reducing component comprises a polystyrene polymer. In some embodiments, the polystyrene sulfonic acid salt is sodium polystyrene sulfonate. In some embodiments, the polystyrene sulfonic acid salt is present in an amount of from about 1 wt % to about 5 wt %, with respect to a total weight of the shelf-stable water-soluble film. In some embodiments, the polystyrene sulfonic acid salt has a molecular weight of from about 50,000 g/mol to about 100,000 g/mol. In some embodiments, the polystyrene sulfonic acid salt has a molecular weight of about 70,000 g/mol. In some embodiments, the polysaccharide is alginic acid or a salt thereof. In some embodiments, the alginic acid salt is sodium alginate. In some embodiments, the shelf-stable water-soluble film comprises iso-tridecyl alcohol.

Also disclosed herein are shelf-stable water-soluble films comprising: (a) a hydrophilic polymer blend with a polymeric resin weight average molecular weight of from about 20,000 g/mol to about 80,000 g/mol, wherein the hydrophilic polymer blend comprises: (i) a polyoxazoline polymer, (ii) a polysaccharide, or (iii) a polypeptide; and (b) water content of at least about 1 wt %, with respect to a total weight of the shelf-stable water-soluble film; wherein the shelf-stable water-soluble film at a thickness of about 3 mil when contacted with water at 26° C. has a disintegration time of less than about 60 seconds, and wherein the shelf-stable water-soluble film has a tensile strength of from about 10 Mpa to about 30 Mpa, as determined by a universal tensile tester. In some embodiments, the shelf-stable water-soluble film has an elongation at break of at least about 500%. In some embodiments, the shelf-stable water-soluble film at a thickness of about 3 mil has a shorter disintegration time when contacted with water at 26° C., relative to a disintegration time under the same conditions of an otherwise comparable film that does not comprise the polyoxazoline polymer, the polysaccharide, or the polypeptide. In some embodiments, the hydrophilic polymer blend further comprises a polyvinyl alcohol polymer. In some embodiments, the polyvinyl alcohol polymer is present in a total amount of from about 50% to about 80% by weight, with respect to a total weight of the shelf-stable water-soluble film. In some embodiments, the polyvinyl alcohol polymer has a net degree of hydrolysis of from about 75% to about 90%. In some embodiments, the water content is from about 1 wt % to about 10 wt %, with respect to the total weight of the shelf-stable water-soluble film. In some embodiments, the hydrophilic polymer blend comprises the polypeptide. In some embodiments, the polypeptide is casein, zein, wheat gluten, whey protein, soy protein, collagen or gelatin. In some embodiments, the hydrophilic polymer blend comprises the polysaccharide. In some embodiments, the polysaccharide is selected from the group consisting of: hydroxypropyl methyl cellulose, carboxymethyl cellulose, methyl ether cellulose, microcrystalline cellulose, chitosan, pullulan, methoxyl pectin, curdlan, ceral beta glucan, gellan, konjac glucomannan, and polydextrose, carrageenan, laminarin, alginic acid, mannan, mannitol, porphyrin, xylan, and a salt of any of these. In some embodiments, the hydrophilic polymer blend comprises the polyoxazoline polymer. In some embodiments, the polyoxazoline polymer is poly(2-ethyl-2-oxazoline). In some embodiments, the poly(2-ethyl-2-oxazoline) is present in an amount of from about 1 wt % to about 25 wt %, with respect to a total weight of the shelf-stable water-soluble film. In some embodiments, the shelf-stable water-soluble film further comprises from about 0.5 wt % to about 5 wt % of a small molecular weight polyhydric alcohol, with respect to a total weight of the shelf-stable water-soluble film. In some embodiments, the small molecular weight polyhydric alcohol has a molecular weight of less than 200 g/mol. In some embodiments, the hydrophilic polymer blend further comprises polyvinylpyrrolidone. In some embodiments, the polyvinylpyrrolidone is present in an amount of from about 1 wt % to about 10 wt %, with respect to a total weight of the shelf-stable water-soluble film. In some embodiments, the polyvinylpyrrolidone has a molecular weight of from about 20,000 g/mol to about 400,000 g/mol. In some embodiments, the shelf-stable water-soluble film further comprises a bubble-reducing component that is present in an amount sufficient to reduce formation of bubbles in the shelf-stable water-soluble film, as compared to an otherwise-comparable film that does not comprise the bubble-reducing component, as determined by heating the shelf-stable water-soluble film and the otherwise comparable film and visualizing the shelf-stable water-soluble film and the otherwise comparable film after cooling. In some embodiments, the bubble-reducing component comprises a polystyrene polymer. In some embodiments, the polystyrene sulfonic acid salt is sodium polystyrene sulfonate. In some embodiments, the polystyrene sulfonic acid salt is present in an amount of from about 1 wt % to about 5 wt %, with respect to a total weight of the shelf-stable water-soluble film. In some embodiments, the polystyrene sulfonic acid salt has a molecular weight of from about 50,000 g/mol to about 100,000 g/mol. In some embodiments, the polystyrene sulfonic acid salt has a molecular weight of about 70,000 g/mol. In some embodiments, the polysaccharide is alginic acid or a salt thereof. In some embodiments, the alginic acid salt is sodium alginate. In some embodiments, the shelf-stable water-soluble film comprises iso-tridecyl alcohol.

Also disclosed herein are compositions comprising a shelf-stable water-soluble film described herein. In some embodiments, the composition further comprises a hygiene product. In some embodiments, the hygiene product is a shampoo, a conditioner, or a body wash. In some embodiments, the hygiene product comprises a total water content of less than about 25 wt %, with respect to a total weight of the water-containing liquid hygiene product. In some embodiments, the hygiene product comprises a polyol in an amount ranging from about 5 wt % to about 35 wt %, wherein the polyol is at least one of ethylene glycol, poly(ethylene glycol), butylene glycol, hexylene glycol, propylene glycol, dipropylene glycol, or glycerin. In some embodiments, the hygiene product comprise surfactants in an amount ranging from about 5 wt % to about 50 wt %. In some embodiments, the hygiene product comprises at least one polyglyceryl fatty acid ester (PGE) selected from the group consisting of polyglyceryl-2 caprate, polyglyceryl-3 caprate, polyglyceryl-3 laurate, and polyglyceryl-6 esters. In some embodiments, the composition further comprises an active ingredient. In some embodiments, the active ingredient is a moisturizer, a vitamin, an oil, retinoic acid, retinol, hyaluronic acid, an alpha-hydroxy acid, a poly-hydroxy acid, a beta-hydroxy acid, ceramide, green tea extract, an algae extract, salicylic acid, linoleic acid, azelaic acid, kaolin clay, CoQ10, lactic acid, centella asiatica, niacinamide, caffeine, glycolic acid, licorice, benzoyl peroxide, glutathione, bakuchiol, mandelic acid, squalene, snail gel, arbutin, aloe vera, honey, biotin, seaweed, tapioca, charcoal, shea butter, cocoa butter, or a combination thereof. In some embodiments, the vitamin is vitamin E, vitamin C, vitamin B3, or a combination thereof. In some embodiments, the oil is argan oil, avocado oil, tea tree oil, olive oil, castor oil, sunflower oil, lavender oil, rosemary oil, cedar wood oil, jojoba oil, thyme oil, peppermint oil, chamomile oil, sage oil, lemon oil, patchouli oil, tea tree oil, ylang oil, acai oil, vetiver oil, carrot seed oil, cypress oil, helichrysum oil, hemp seed oil, rosehip oil, or a combination thereof. In some embodiments, the composition further comprises a cleaning agent. In some embodiments, the cleaning agent is a household care product. In some embodiments, the household care product comprises an ethoxylated-propoxylated alcohol, an epoxy-capped poly(oxyalkylated) alcohol, an ether-capped poly(oxyalkylated) alcohol surfactant, a block polyoxyethylene-polyoxypropylene polymeric compound, a C12-C20 alkyl amine oxide, an alkyl amphocarboxylic surfactant, a betaine, a sultaine, or a combination thereof. In some embodiments, the household care product comprises an enzyme. In some embodiments, the enzyme is a hemicellulase, a peroxidase, a protease, a cellulase, a xylanase, a lipase, a phospholipase, an esterase, a cutinase, a pectinase, a mannanase, a pectate lyase, a keratinase, a reductase, an oxidase, a phenoloxidase, a lipoxygenase, a ligninase, a pullulanase, a tannase, a pentosanase, a malanase, a β-glucanase, an arabinosidase, a hyaluronidase, a chondroitinase, a laccase, an amylase, or a combination thereof. In some embodiments, household care product comprises glycerol, propylene glycol, isopropanol, Marlipal C₁₂₋₁₄EO₇, C₁₃₋₁₅EO₉, C₉₋₁₁EO₉, alhylbenzene, alkylbenzenesulfonate, alkylsulfate, dimethyl hydroxyethylammonium chloride, MEA-LAS, MEA-Laureth Sulfate, PEI ethoxylate, C₁₂₋₁₆ Paeth, C₁₄₋₁₅ Pareth, C₁₀₋₁₆ Pareth, propylene glycol, acrylamide, bentonite, benzisothiozolinone, C₁₀₋₁₆ alkyldimethylanine oxide, ethanolamine citrate, ethanolamine laurate, ethanolanine myristate, ethanolanine palintate, ethanolamine oleate, ethanolamine stearate, palmitic acid, tripropylene glycol, or a combination thereof.

Also disclosed herein are hygiene product pods. A hygiene product pod comprises a hygiene product as described herein sealed in a water-soluble envelope made of a shelf-stable water-soluble film as described herein. In some embodiments, a hygiene product pods comprises (a) a water-soluble envelope, wherein the water-soluble envelope comprises a hydrophilic polymer blend that comprises a polyoxazoline polymer or a polypeptide; and (b) a water-containing liquid hygiene product sealed in the water-soluble envelope, wherein the water-containing liquid hygiene product does not phase separate within the water-soluble envelope; and wherein when a group of about 100 hygiene product pods are stored for at least 24 hours at 23° C. and 50% relative humidity, less than 5 of the group of about 100 hygiene product pods degrade, as determined by leakage of the water-containing liquid hygiene product. In some embodiments, the hydrophilic polymer blend further comprises a polyvinyl alcohol polymer. In some embodiments, the polyvinyl alcohol polymer has a net degree of hydrolysis of from about 75% to about 90%. In some embodiments, the polyvinyl alcohol polymer is present in a total amount of from about 50% to about 80% by weight, with respect to a total weight of the hydrophilic polymer blend. In some embodiments, the hydrophilic polymer blend comprises a water content from about 1 wt % to about 10 wt %, with respect to the total weight of the hydrophilic polymer blend. In some embodiments, the hydrophilic polymer blend comprises the polypeptide. In some embodiments, the polypeptide is casein, zein, wheat gluten, whey protein, soy protein, collagen or gelatin. In some embodiments, the hydrophilic polymer blend comprises a polysaccharide. In some embodiments, the polysaccharide is selected from the group consisting of: hydroxypropyl methyl cellulose, carboxymethyl cellulose, methyl ether cellulose, microcrystalline cellulose, chitosan, pullulan, methoxyl pectin, curdlan, ceral beta glucan, gellan, konjac glucomannan, polydextrose, carrageenan, laminarin, alginic acid, mannan, mannitol, porphyrin, xylan, and a salt of any of these. In some embodiments, the hydrophilic polymer blend comprises the polyoxazoline polymer. In some embodiments, the polyoxazoline polymer is poly(2-ethyl-2-oxazoline). In some embodiments, the poly(2-ethyl-2-oxazoline has a molecular weight of at least 20,000 g/mol. In some embodiments, the poly(2-ethyl-2-oxazoline) is present in an amount of from about 1 wt % to about 25 wt %, with respect to a total weight of the hydrophilic polymer blend. In some embodiments, the hydrophilic polymer blend has a polymeric resin weight average molecular weight of from about 20,000 g/mol to about 80,000 g/mol. In some embodiments, the hydrophilic polymer blend comprises from about 0.5 wt % to about 5 wt % of a polyhydric alcohol with a molecular weight of less than 200 g/mol, with respect to a total weight of the hydrophilic polymer blend. In some embodiments, the hydrophilic polymer blend further comprises polyvinylpyrrolidone. In some embodiments, the polyvinylpyrrolidone is present in an amount of from about 1 wt % to about 10 wt %, with respect to a total weight of the shelf-stable water-soluble film. In some embodiments, the polyvinylpyrrolidone has a molecular weight of from about 20,000 g/mol to about 400,000 g/mol. In some embodiments, the hydrophilic polymer blend further comprises a bubble-reducing component that is present in an amount sufficient to reduce formation of bubbles in the water-soluble envelope, as compared to an otherwise-comparable water-soluble envelope that does not comprise the bubble-reducing component, as determined by heating the water-soluble envelope and the otherwise comparable water-soluble envelope and visualizing the water-soluble envelope and the otherwise comparable water-soluble envelope after cooling. In some embodiments, the bubble-reducing component comprises a polystyrene polymer. In some embodiments, the polystyrene sulfonic acid salt is sodium polystyrene sulfonate. In some embodiments, the polystyrene sulfonic acid salt is present in an amount of from about 1 wt % to about 5 wt %, with respect to a total weight of the shelf-stable water-soluble film. In some embodiments, the polystyrene sulfonic acid salt has a molecular weight of from about 50,000 g/mol to about 100,000 g/mol. In some embodiments, the polystyrene sulfonic acid salt has a molecular weight of about 70,000 g/mol. In some embodiments, the polysaccharide is alginic acid or a salt thereof. In some embodiments, the alginic acid salt is sodium alginate. In some embodiments, the hydrophilic polymer blend comprises iso-tridecyl alcohol. In some embodiments, the water-containing liquid hygiene product is a shampoo, a conditioner, a body wash, or a shaving cream. In some embodiments, the water-containing liquid hygiene product comprises a total water content of less than about 25 wt %, with respect to a total weight of the water-containing liquid hygiene product. In some embodiments, the water-containing liquid hygiene product comprises a polyol in an amount ranging from about 5 wt % to about 35 wt %, wherein the polyol is at least one of ethylene glycol, poly(ethylene glycol), butylene glycol, hexylene glycol, propylene glycol, dipropylene glycol, or glycerin. In some embodiments, the water-containing liquid hygiene product comprise surfactants in an amount ranging from about 5 wt % to about 50 wt %. In some embodiments, the water-containing liquid hygiene product comprises at least one polyglyceryl fatty acid ester (PGE) selected from the group consisting of polyglyceryl-2 caprate, polyglyceryl-3 caprate, polyglyceryl-3 laurate, and polyglyceryl-6 esters.

Also disclosed herein are hygiene product pods comprising: (a) a water-soluble envelope, wherein the water-soluble envelope comprises: (i) a hydrophilic polymer blend with a polymeric resin weight average molecular weight of from about 20,000 g/mol to about 80,000 g/mol, wherein the hydrophilic polymer blend comprises: (1) poly(2-ethyl-2-oxazoline) at a molecular weight of at least 20,000 g/mol, and (2) a blend of at least two polyvinyl alcohol polymers that have a net degree of hydrolysis of from about 75% to about 90%, (ii) water content of from about 1 wt % to about 10 wt %, with respect to a total weight of the water-soluble envelope, and (iii) less than about 5 wt % of a polyhydric alcohol with a molecular weight less than about 200 g/mol, with respect to a total weight of the shelf-stable water-soluble film; (b) a water-containing liquid hygiene product sealed in the water-soluble envelope, wherein the water-containing liquid hygiene product comprises, based on a total weight of the water-containing liquid hygiene product: (i) a non-aqueous carrier comprising one or more polyol solvents in an amount ranging from about 10 wt % to about 30 wt %, and (ii) water content of less than about 25 wt %, wherein when a group of about 100 hygiene product pods are stored for at least 24 hours at 23° C. and 50% relative humidity, less than 5 of the group of about 100 hygiene product pods degrade, as determined by leakage of the water-containing liquid hygiene product. In some embodiments, the water containing liquid hygiene product further comprises a polyglyceryl fatty acid ester (PGE) chassis in an amount ranging up to 50 wt %, based on a total weight of the water-containing liquid hygiene product.

Also disclosed herein are hygiene product pods comprising: (a) a water-soluble envelope, wherein the water-soluble envelope comprises: (i) a hydrophilic polymer blend with a polymeric resin weight average molecular weight of from about 20,000 g/mol to about 80,000 g/mol, wherein the hydrophilic polymer blend comprises: (1) poly(2-ethyl-2-oxazoline) at a molecular weight of at least 20,000 g/mol, and (2) a blend of at least two polyvinyl alcohol polymers that have a net degree of hydrolysis of from about 75% to about 90%, (ii) water content of from about 1 wt % to about 10 wt %, with respect to a total weight of the water-soluble envelope, and (iii) less than about 5 wt % of a polyhydric alcohol with a molecular weight less than about 200 g/mol, with respect to a total weight of the shelf-stable water-soluble film; (b) a water-containing liquid hygiene product sealed in the water-soluble envelope, wherein the water-containing liquid hygiene product comprises, based on a total weight of the water-containing liquid hygiene product: (i) a non-aqueous carrier comprising one or more polyol solvents in an amount ranging from about 10 wt % to about 30 wt %, (ii) a polyglyceryl fatty acid ester (PGE) chassis in an amount ranging from about 0 wt % to about 50 wt %, and (iii) water content of less than about 25 wt %, wherein when a group of about 100 hygiene product pods are stored for at least 24 hours at 23° C. and 50% relative humidity, less than 5 of the group of about 100 hygiene product pods degrade, as determined by leakage of the water-containing liquid hygiene product.

Also disclosed herein are methods of making a shelf-stable water-soluble hygiene product pod. In some embodiments, a method of making a shelf-stable water-soluble hygiene product pod comprises: (a) dissolving a hydrophilic polymer blend comprising a polyoxazoline polymer or a polypeptide in water; (b) heating the hydrophilic polymer blend, thereby generating a polymer blend film; and encapsulating a liquid hygiene product in the polymer film, thereby generating the shelf-stable water-soluble hygiene product pod.

### DETAILED DESCRIPTION BRIEF DESCRIPTION OF THE DRAWINGS

Novel features of exemplary embodiments are set forth with particularity in the appended claims. A better understanding of the features and advantages will be obtained by reference to the following detailed description that sets forth illustrative embodiments, in which the principles of the disclosed systems and methods are utilized, and the accompanying drawings of which:

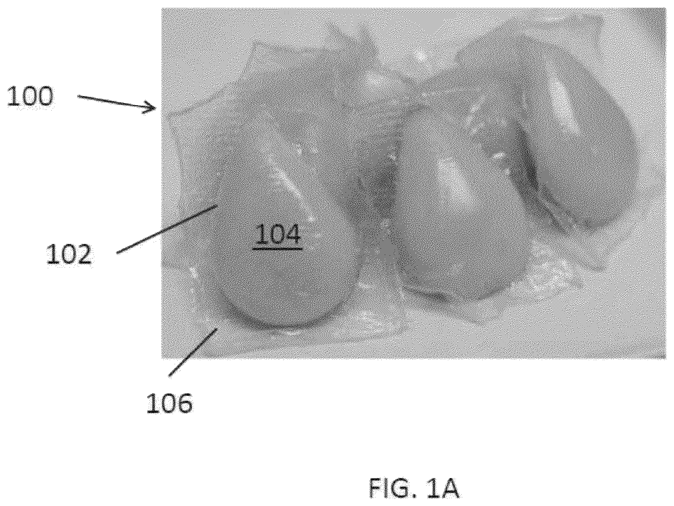

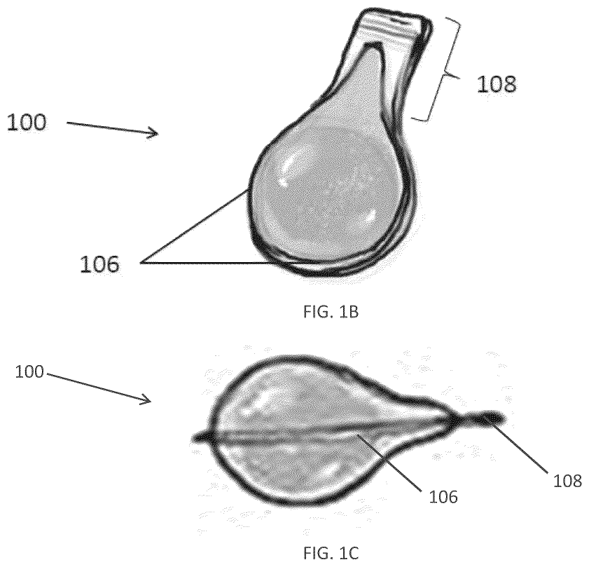

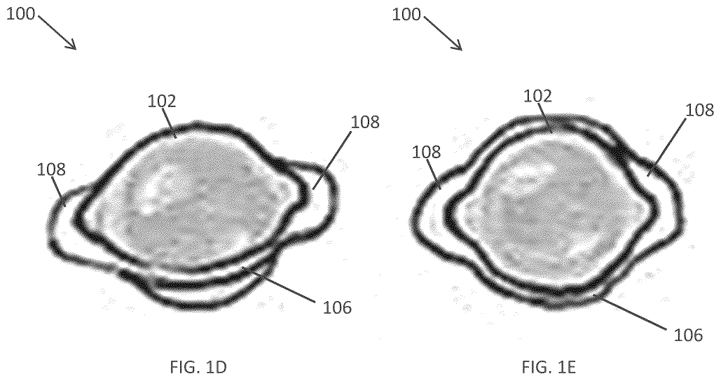

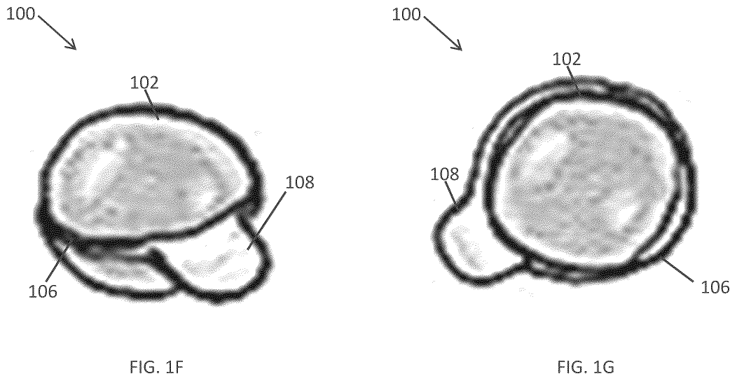

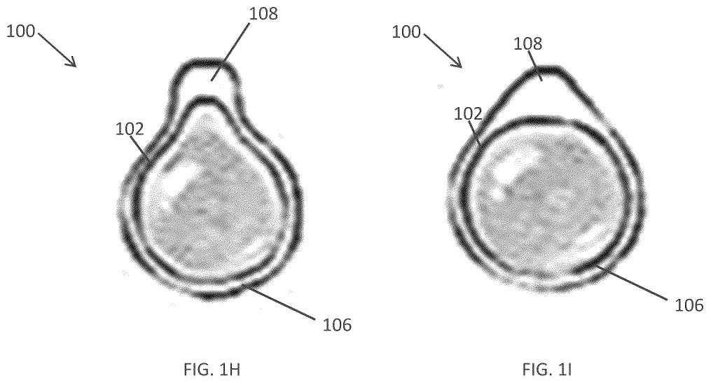

FIGS. 1A-1J depict hygiene product pods as described herein. FIG. 1A shows a photograph of a hygiene product pod, in accordance with embodiments. FIG. 1B shows a perspective front view of a hygiene product pod following an optional cutting process, in accordance with embodiments. FIG. 1C shows a side view of the hygiene product pod of FIG. 1B, in accordance with embodiments. FIG. 1D shows a perspective front view of a hygiene product pod, in accordance with embodiments. FIG. 1E shows a top view of the hygiene product pod of FIG. 1D, in accordance with embodiments. FIG. 1F shows a perspective front view of a hygiene product pod, in accordance with embodiments. FIG. 1G shows a top view of the hygiene product pod of FIG. 1F, in accordance with embodiments. FIG. 1H shows a perspective front view of a hygiene product pod, in accordance with embodiments. FIG. 1I shows a perspective front view of a hygiene product pod, in accordance with embodiments. FIG. 1J shows a perspective front view of a hygiene product pod, in accordance with embodiments.

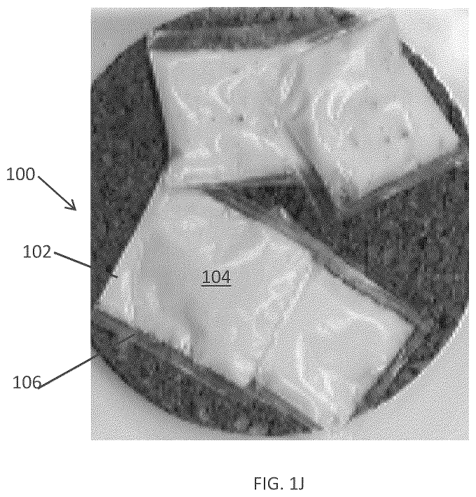

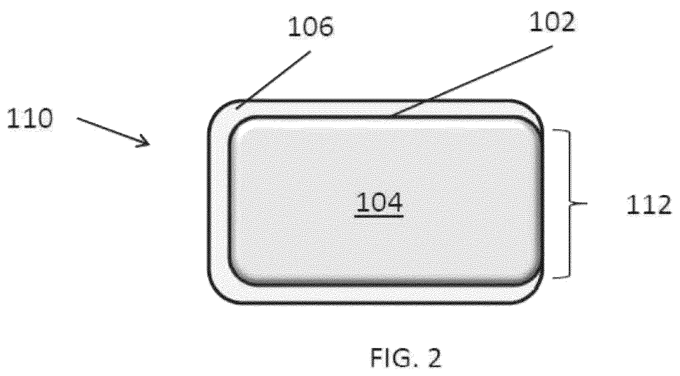

FIG. 2 shows atop plan view of a hygiene product pod, in accordance with embodiments.

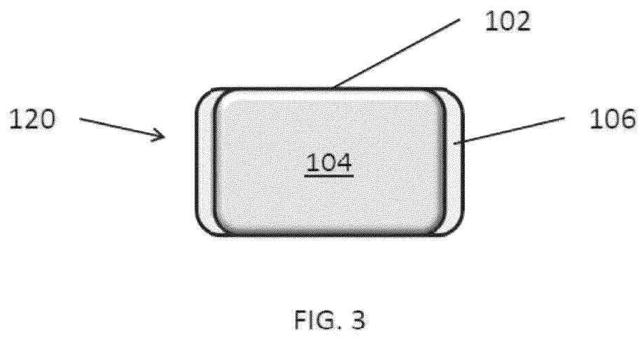

FIG. 3 shows a top view of a hygiene product pod, in accordance with embodiments.

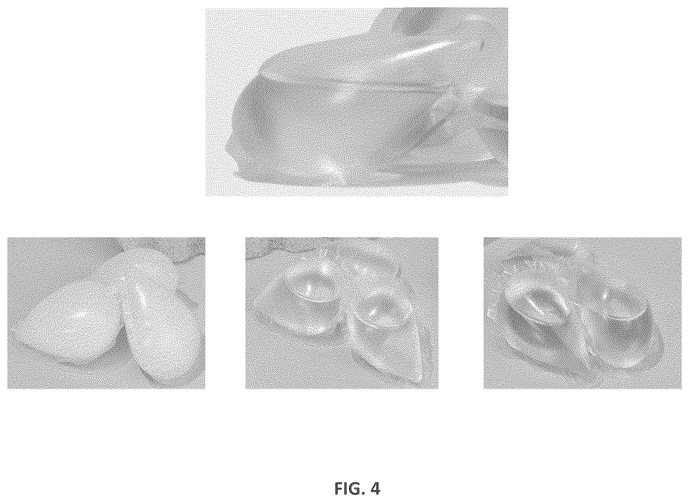

FIG. 4 shows exemplary hygiene product pods, in accordance with embodiments.

### OVERVIEW

Disclosed herein are shelf-stable water-soluble films, and methods of making shelf-stable water-soluble films, for use with, for example, hygiene products as described herein. Such shelf-stable water-films have attractive features that make them useful for cosmetic use. For example, shelf-stable water-soluble films described herein have sufficient structural integrity (e.g. superior tensile strength) and shelf stability when stored under ambient conditions to be useful commercially, and yet are sufficiently soluble when contacted with water to enable rapid disintegration and release of a product stored within. Further, the components of the shelf-stable water-soluble film are safe for cosmetic application to, for example, the skin or hair of a person. Critically, the shelf-stable water-soluble film is compatible with hygiene products (both dry/anhydrous and water-containing) when fabricated as a water-soluble envelope containing the hygiene product encapsulated therein. For example, there is minimal partitioning of components between the hygiene product and the water-soluble envelope, and minimal dissolution/disintegration of the water-soluble envelope due to contact with the hygiene product.

Accordingly, also disclosed herein are hygiene product pods, and methods of making hygiene product pods, comprising a water-soluble envelope made of a shelf-stable water-soluble film described herein, and hygiene products encapsulated therein. Embodiments as described herein comprise a liquid hygiene product that: (a) are stable (e.g. do not phase separate when encapsulated in the water-soluble envelope), (b) contain exemplary ingredients that are naturally sourced, and (c) do not dissolve the water-soluble envelope when encapsulated therein. Such pods can be configured for single use to deliver a hygiene product when the pod is contacted with water. Hygiene product pods as described herein have the potential to significantly reduce plastic waste derived from use of comparable single-use hygiene products stored in plastic containers.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” refers to within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “bubble reducing component” as used herein includes an ingredient added to the polymer resin blend to reduce bubble formation in the corresponding film, as compared to an otherwise comparable film lacking the bubble reducing component, as determined by heating the film containing the bubble reducing component and the otherwise comparable film and visualizing both after cooling.

The term “low molecular weight polyhydric alcohol” as used herein refers to those with a molecular weight below 200 g/mol.

The term “compatibility” as used herein refers to the interchange of components between a film and what the film encapsulates.

### Water-Soluble Films

Disclosed herein are shelf-stable water-soluble films. A shelf-stable water-soluble film as described herein can be used in combination with a hygiene product for cosmetic use. However, such a film comprises components that minimize agglomeration while maximizing compatibility with hygiene products described herein. Given the composition of the hygiene products, the present application provides water-soluble films that are sufficiently compatible with hygiene products described herein while providing sufficient water-solubility for use in a hygiene product pod encapsulating the hygiene product.

While plasticizing ingredients can be used to achieve such an outcome, many plasticizing ingredients are incompatible with hygiene products as described herein. For example, use of small molecular weight plasticizers, such as small molecular weight polyhydric alcohols like glycerin, propylene glycol, trimethylolpropane and butylene glycol, in amounts greater than about 5 wt % (e.g. greater than about 10 wt %) may not be compatible with hygiene products due to the similarity in their respective overall composition, which can lead to dissolution of the film by the hygiene product pod. Accordingly, some embodiments of shelf-stable water-soluble film described herein do not contain such small molecular weight polyhydric alcohols, while other embodiments contain only minimal amounts of such small molecular weight polyhydric alcohols. As an alternative to formulating films with large amounts of small polyhydric alcohols, the present application provides shelf-stable water-soluble films comprising a hydrophilic polymer blend of polymers and water content of at least 1 wt %, with respect to the total weight of the shelf-stable water-soluble film. In some cases, careful selection of the constituent polymers in the blend and control of the polymeric resin weight average molecular weight of the polymer blend can be used to generate a hydrophilic polymer resin with reduced crystallinity, and therefore enhanced solubility in water, relative to comparable films.

### Core Polymer Blend

A polymer blend as described herein can include, for example, a polyoxazoline polymer, a polysaccharide, a polypeptide, and can further include a polyvinyl alcohol polymer, a pyrrolidone polymer, and the like. To this blend can be added additional polymers as described herein sufficient to achieve reduced crystallinity of the shelf-stable water-soluble blend and improve other properties of the film (e.g. reduce agglomeration, dissolution time, or disintegration time of the film when contacted with water). Such additional polymers include, for example, polyacrylic acid, polyalkylene oxides, polycarboxylic acids, polyamides, polyacrylamides, methylmethacrylate copolymers, and natural gums such as xanthan and carrageenans.

In some embodiments, a hydrophilic polymer blend has a weight average molecular weight of from about 10000 g/mol to about 20000 g/mol, from about 10000 g/mol to about 21000 g/mol, from about 10000 g/mol to about 22000 g/mol, from about 10000 g/mol to about 23000 g/mol, from about 10000 g/mol to about 24000 g/mol, from about 10000 g/mol to about 25000 g/mol, from about 10000 g/mol to about 26000 g/mol, from about 10000 g/mol to about 27000 g/mol, from about 10000 g/mol to about 28000 g/mol, from about 10000 g/mol to about 29000 g/mol, from about 10000 g/mol to about 30000 g/mol, from about 10000 g/mol to about 31000 g/mol, from about 10000 g/mol to about 32000 g/mol, from about 10000 g/mol to about 33000 g/mol, from about 10000 g/mol to about 34000 g/mol, from about 10000 g/mol to about 35000 g/mol, from about 10000 g/mol to about 36000 g/mol, from about 10000 g/mol to about 37000 g/mol, from about 10000 g/mol to about 38000 g/mol, from about 10000 g/mol to about 39000 g/mol, from about 10000 g/mol to about 40000 g/mol, from about 10000 g/mol to about 41000 g/mol, from about 10000 g/mol to about 42000 g/mol, from about 10000 g/mol to about 43000 g/mol, from about 10000 g/mol to about 44000 g/mol, from about 10000 g/mol to about 45000 g/mol, from about 10000 g/mol to about 46000 g/mol, from about 10000 g/mol to about 47000 g/mol, from about 10000 g/mol to about 48000 g/mol, from about 10000 g/mol to about 49000 g/mol, from about 10000 g/mol to about 50000 g/mol, from about 10000 g/mol to about 51000 g/mol, from about 10000 g/mol to about 52000 g/mol, from about 10000 g/mol to about 53000 g/mol, from about 10000 g/mol to about 54000 g/mol, from about 10000 g/mol to about 55000 g/mol, from about 10000 g/mol to about 56000 g/mol, from about 10000 g/mol to about 57000 g/mol, from about 10000 g/mol to about 58000 g/mol, from about 10000 g/mol to about 59000 g/mol, from about 10000 g/mol to about 60000 g/mol, from about 10000 g/mol to about 61000 g/mol, from about 10000 g/mol to about 62000 g/mol, from about 10000 g/mol to about 63000 g/mol, from about 10000 g/mol to about 64000 g/mol, from about 10000 g/mol to about 65000 g/mol, from about 10000 g/mol to about 66000 g/mol, from about 10000 g/mol to about 67000 g/mol, from about 10000 g/mol to about 68000 g/mol, from about 10000 g/mol to about 69000 g/mol, from about 10000 g/mol to about 70000 g/mol, from about 10000 g/mol to about 71000 g/mol, from about 10000 g/mol to about 72000 g/mol, from about 10000 g/mol to about 73000 g/mol, from about 10000 g/mol to about 74000 g/mol, from about 10000 g/mol to about 75000 g/mol, from about 10000 g/mol to about 76000 g/mol, from about 10000 g/mol to about 77000 g/mol, from about 10000 g/mol to about 78000 g/mol, from about 10000 g/mol to about 79000 g/mol, from about 10000 g/mol to about 80000 g/mol, from about 10000 g/mol to about 81000 g/mol, from about 10000 g/mol to about 82000 g/mol, from about 10000 g/mol to about 83000 g/mol, from about 10000 g/mol to about 84000 g/mol, from about 10000 g/mol to about 85000 g/mol, from about 10000 g/mol to about 86000 g/mol, from about 10000 g/mol to about 87000 g/mol, from about 10000 g/mol to about 88000 g/mol, from about 10000 g/mol to about 89000 g/mol, from about 10000 g/mol to about 90000 g/mol, from about 10000 g/mol to about 91000 g/mol, from about 10000 g/mol to about 92000 g/mol, from about 10000 g/mol to about 93000 g/mol, from about 10000 g/mol to about 94000 g/mol, from about 10000 g/mol to about 95000 g/mol, from about 10000 g/mol to about 96000 g/mol, from about 10000 g/mol to about 97000 g/mol, from about 10000 g/mol to about 98000 g/mol, from about 10000 g/mol to about 99000 g/mol, from about 10000 g/mol to about 100000 g/mol, from about 10000 g/mol to about 101000 g/mol, from about 10000 g/mol to about 102000 g/mol, from about 10000 g/mol to about 103000 g/mol, from about 10000 g/mol to about 104000 g/mol, from about 10000 g/mol to about 105000 g/mol, from about 10000 g/mol to about 106000 g/mol, from about 10000 g/mol to about 107000 g/mol, from about 10000 g/mol to about 108000 g/mol, from about 10000 g/mol to about 109000 g/mol, from about 10000 g/mol to about 110000 g/mol, from about 10000 g/mol to about 111000 g/mol, from about 10000 g/mol to about 112000 g/mol, from about 10000 g/mol to about 113000 g/mol, from about 10000 g/mol to about 114000 g/mol, from about 10000 g/mol to about 115000 g/mol, from about 10000 g/mol to about 116000 g/mol, from about 10000 g/mol to about 117000 g/mol, from about 10000 g/mol to about 118000 g/mol, from about 10000 g/mol to about 119000 g/mol, from about 10000 g/mol to about 120000 g/mol, from about 10000 g/mol to about 121000 g/mol, from about 10000 g/mol to about 122000 g/mol, from about 10000 g/mol to about 123000 g/mol, from about 10000 g/mol to about 124000 g/mol, from about 10000 g/mol to about 125000 g/mol, from about 10000 g/mol to about 126000 g/mol, from about 10000 g/mol to about 127000 g/mol, from about 10000 g/mol to about 128000 g/mol, from about 10000 g/mol to about 129000 g/mol, from about 10000 g/mol to about 130000 g/mol, from about 10000 g/mol to about 131000 g/mol, from about 10000 g/mol to about 132000 g/mol, from about 10000 g/mol to about 133000 g/mol, from about 10000 g/mol to about 134000 g/mol, from about 10000 g/mol to about 135000 g/mol, from about 10000 g/mol to about 136000 g/mol, from about 10000 g/mol to about 137000 g/mol, from about 10000 g/mol to about 138000 g/mol, from about 10000 g/mol to about 139000 g/mol, from about 10000 g/mol to about 140000 g/mol, from about 10000 g/mol to about 141000 g/mol, from about 10000 g/mol to about 142000 g/mol, from about 10000 g/mol to about 143000 g/mol, from about 10000 g/mol to about 144000 g/mol, from about 10000 g/mol to about 145000 g/mol, from about 10000 g/mol to about 146000 g/mol, from about 10000 g/mol to about 147000 g/mol, from about 10000 g/mol to about 148000 g/mol, from about 10000 g/mol to about 149000 g/mol, from about 10000 g/mol to about 150000 g/mol, from about 10000 g/mol to about 151000 g/mol, from about 10000 g/mol to about 152000 g/mol, from about 10000 g/mol to about 153000 g/mol, from about 10000 g/mol to about 154000 g/mol, from about 10000 g/mol to about 155000 g/mol, from about 10000 g/mol to about 156000 g/mol, from about 10000 g/mol to about 157000 g/mol, from about 10000 g/mol to about 158000 g/mol, from about 10000 g/mol to about 159000 g/mol, from about 10000 g/mol to about 160000 g/mol, from about 10000 g/mol to about 161000 g/mol, from about 10000 g/mol to about 162000 g/mol, from about 10000 g/mol to about 163000 g/mol, from about 10000 g/mol to about 164000 g/mol, from about 10000 g/mol to about 165000 g/mol, from about 10000 g/mol to about 166000 g/mol, from about 10000 g/mol to about 167000 g/mol, from about 10000 g/mol to about 168000 g/mol, from about 10000 g/mol to about 169000 g/mol, from about 10000 g/mol to about 170000 g/mol, from about 10000 g/mol to about 171000 g/mol, from about 10000 g/mol to about 172000 g/mol, from about 10000 g/mol to about 173000 g/mol, from about 10000 g/mol to about 174000 g/mol, from about 10000 g/mol to about 175000 g/mol, from about 10000 g/mol to about 176000 g/mol, from about 10000 g/mol to about 177000 g/mol, from about 10000 g/mol to about 178000 g/mol, from about 10000 g/mol to about 179000 g/mol, from about 10000 g/mol to about 180000 g/mol, from about 10000 g/mol to about 181000 g/mol, from about 10000 g/mol to about 182000 g/mol, from about 10000 g/mol to about 183000 g/mol, from about 10000 g/mol to about 184000 g/mol, from about 10000 g/mol to about 185000 g/mol, from about 10000 g/mol to about 186000 g/mol, from about 10000 g/mol to about 187000 g/mol, from about 10000 g/mol to about 188000 g/mol, from about 10000 g/mol to about 189000 g/mol, from about 10000 g/mol to about 190000 g/mol, from about 10000 g/mol to about 191000 g/mol, from about 10000 g/mol to about 192000 g/mol, from about 10000 g/mol to about 193000 g/mol, from about 10000 g/mol to about 194000 g/mol, from about 10000 g/mol to about 195000 g/mol, from about 10000 g/mol to about 196000 g/mol, from about 10000 g/mol to about 197000 g/mol, from about 10000 g/mol to about 198000 g/mol, from about 10000 g/mol to about 199000 g/mol, or from about 10000 g/mol to about 200000 g/mol. In some embodiments, a hydrophilic polymer blend has a weight average molecular weight of from about 10000 g/mol to about 100000 g/mol, from about 20000 g/mol to about 80000, or from about 30000 g/mol to about 50000 g/mol. In some embodiments, a hydrophilic polymer blend has a weight average molecular weight of at least about 5000 g/mol, 6000 g/mol, 7000 g/mol, 8000 g/mol, 9000 g/mol, 10000 g/mol, 11000 g/mol, 12000 g/mol, 13000 g/mol, 14000 g/mol, 15000 g/mol, 16000 g/mol, 17000 g/mol, 18000 g/mol, 19000 g/mol, 20000 g/mol, 21000 g/mol, 22000 g/mol, 23000 g/mol, 24000 g/mol, 25000 g/mol, 26000 g/mol, 27000 g/mol, 28000 g/mol, 29000 g/mol, 30000 g/mol, 31000 g/mol, 32000 g/mol, 33000 g/mol, 34000 g/mol, 35000 g/mol, 36000 g/mol, 37000 g/mol, 38000 g/mol, 39000 g/mol, 40000 g/mol, 41000 g/mol, 42000 g/mol, 43000 g/mol, 44000 g/mol, 45000 g/mol, 46000 g/mol, 47000 g/mol, 48000 g/mol, 49000 g/mol, 50000 g/mol, 51000 g/mol, 52000 g/mol, 53000 g/mol, 54000 g/mol, 55000 g/mol, 56000 g/mol, 57000 g/mol, 58000 g/mol, 59000 g/mol, 60000 g/mol, 61000 g/mol, 62000 g/mol, 63000 g/mol, 64000 g/mol, 65000 g/mol, 66000 g/mol, 67000 g/mol, 68000 g/mol, 69000 g/mol, 70000 g/mol, 71000 g/mol, 72000 g/mol, 73000 g/mol, 74000 g/mol, 75000 g/mol, 76000 g/mol, 77000 g/mol, 78000 g/mol, 79000 g/mol, 80000 g/mol, 81000 g/mol, 82000 g/mol, 83000 g/mol, 84000 g/mol, 85000 g/mol, 86000 g/mol, 87000 g/mol, 88000 g/mol, 89000 g/mol, 90000 g/mol, 91000 g/mol, 92000 g/mol, 93000 g/mol, 94000 g/mol, 95000 g/mol, 96000 g/mol, 97000 g/mol, 98000 g/mol, 99000 g/mol, 100000 g/mol, 101000 g/mol, 102000 g/mol, 103000 g/mol, 104000 g/mol, 105000 g/mol, 106000 g/mol, 107000 g/mol, 108000 g/mol, 109000 g/mol, 110000 g/mol, 111000 g/mol, 112000 g/mol, 113000 g/mol, 114000 g/mol, 115000 g/mol, 116000 g/mol, 117000 g/mol, 118000 g/mol, 119000 g/mol, 120000 g/mol, 121000 g/mol, 122000 g/mol, 123000 g/mol, 124000 g/mol, 125000 g/mol, 126000 g/mol, 127000 g/mol, 128000 g/mol, 129000 g/mol, 130000 g/mol, 131000 g/mol, 132000 g/mol, 133000 g/mol, 134000 g/mol, 135000 g/mol, 136000 g/mol, 137000 g/mol, 138000 g/mol, 139000 g/mol, 140000 g/mol, 141000 g/mol, 142000 g/mol, 143000 g/mol, 144000 g/mol, 145000 g/mol, 146000 g/mol, 147000 g/mol, 148000 g/mol, 149000 g/mol, 150000 g/mol, 151000 g/mol, 152000 g/mol, 153000 g/mol, 154000 g/mol, 155000 g/mol, 156000 g/mol, 157000 g/mol, 158000 g/mol, 159000 g/mol, 160000 g/mol, 161000 g/mol, 162000 g/mol, 163000 g/mol, 164000 g/mol, 165000 g/mol, 166000 g/mol, 167000 g/mol, 168000 g/mol, 169000 g/mol, 170000 g/mol, 171000 g/mol, 172000 g/mol, 173000 g/mol, 174000 g/mol, 175000 g/mol, 176000 g/mol, 177000 g/mol, 178000 g/mol, 179000 g/mol, 180000 g/mol, 181000 g/mol, 182000 g/mol, 183000 g/mol, 184000 g/mol, 185000 g/mol, 186000 g/mol, 187000 g/mol, 188000 g/mol, 189000 g/mol, 190000 g/mol, 191000 g/mol, 192000 g/mol, 193000 g/mol, 194000 g/mol, 195000 g/mol, 196000 g/mol, 197000 g/mol, 198000 g/mol, 199000 g/mol, or 200000 g/mol.

In some embodiments, a hydrophilic polymer blend includes a polyoxazoline polymer. Such polyoxazoline polymers include poly(2-alkyl-2-oxazolines) such as poly(2-methyl-2-oxazoline), poly(2-ethyl-2-oxazoline), or poly(2-isopropyl-2-oxazoline), poly(2-cyclopropyl-2-oxazoline), poly(2-n-propyl-2-oxazoline); and poly(2-amino-2-oxazolines) such as poly(2-dimethylamino-2-oxazoline), poly(2-ethylamino-2-oxazoline), poly(2-diethylamino-2-oxazoline), or poly(2-diisopropylamino-2-oxazoline). In some specific cases, a polyoxazoline polymer is Aquazol 50. In some cases, a polyoxazoline polymer is present in an amount of from about 1 wt % to about 50 wt %, from about 1 wt % to about 49 wt %, from about 1 wt % to about 48 wt %, from about 1 wt % to about 47 wt %, from about 1 wt % to about 46 wt %, from about 1 wt % to about 45 wt %, from about 1 wt % to about 44 wt %, from about 1 wt % to about 43 wt %, from about 1 wt % to about 42 wt %, from about 1 wt % to about 41 wt %, from about 1 wt % to about 40 wt %, from about 1 wt % to about 39 wt %, from about 1 wt % to about 38 wt %, from about 1 wt % to about 37 wt %, from about 1 wt % to about 36 wt %, from about 1 wt % to about 35 wt %, from about 1 wt % to about 34 wt %, from about 1 wt % to about 33 wt %, from about 1 wt % to about 32 wt %, from about 1 wt % to about 31 wt %, from about 1 wt % to about 30 wt %, from about 1 wt % to about 29 wt %, from about 1 wt % to about 28 wt %, from about 1 wt % to about 27 wt %, from about 1 wt % to about 26 wt %, from about 1 wt % to about 25 wt %, from about 1 wt % to about 24 wt %, from about 1 wt % to about 23 wt %, from about 1 wt % to about 22 wt %, from about 1 wt % to about 21 wt %, from about 1 wt % to about 20 wt %, from about 1 wt % to about 19 wt %, from about 1 wt % to about 18 wt %, from about 1 wt % to about 17 wt %, from about 1 wt % to about 16 wt %, from about 1 wt % to about 15 wt %, from about 1 wt % to about 14 wt %, from about 1 wt % to about 13 wt %, from about 1 wt % to about 12 wt %, from about 1 wt % to about 11 wt %, from about 1 wt % to about 10 wt %, from about 1 wt % to about 9 wt %, from about 1 wt % to about 8 wt %, from about 1 wt % to about 7 wt %, from about 1 wt % to about 6 wt %, from about 1 wt % to about 5 wt %. In some embodiments, a polyoxazoline polymer is present in an amount of at least about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, or greater than about 90 wt %.

In some embodiments, a hydrophilic polymer blend includes a polypeptide. Such polypeptides include casein, zein, wheat gluten, whey protein, soy protein, collagen and gelatin. In some cases, a polypeptide is present in an amount of from about 1 wt % to about 50 wt %, from about 1 wt % to about 49 wt %, from about 1 wt % to about 48 wt %, from about 1 wt % to about 47 wt %, from about 1 wt % to about 46 wt %, from about 1 wt % to about 45 wt %, from about 1 wt % to about 44 wt %, from about 1 wt % to about 43 wt %, from about 1 wt % to about 42 wt %, from about 1 wt % to about 41 wt %, from about 1 wt % to about 40 wt %, from about 1 wt % to about 39 wt %, from about 1 wt % to about 38 wt %, from about 1 wt % to about 37 wt %, from about 1 wt % to about 36 wt %, from about 1 wt % to about 35 wt %, from about 1 wt % to about 34 wt %, from about 1 wt % to about 33 wt %, from about 1 wt % to about 32 wt %, from about 1 wt % to about 31 wt %, from about 1 wt % to about 30 wt %, from about 1 wt % to about 29 wt %, from about 1 wt % to about 28 wt %, from about 1 wt % to about 27 wt %, from about 1 wt % to about 26 wt %, from about 1 wt % to about 25 wt %, from about 1 wt % to about 24 wt %, from about 1 wt % to about 23 wt %, from about 1 wt % to about 22 wt %, from about 1 wt % to about 21 wt %, from about 1 wt % to about 20 wt %, from about 1 wt % to about 19 wt %, from about 1 wt % to about 18 wt %, from about 1 wt % to about 17 wt %, from about 1 wt % to about 16 wt %, from about 1 wt % to about 15 wt %, from about 1 wt % to about 14 wt %, from about 1 wt % to about 13 wt %, from about 1 wt % to about 12 wt %, from about 1 wt % to about 11 wt %, from about 1 wt % to about 10 wt %, from about 1 wt % to about 9 wt %, from about 1 wt % to about 8 wt %, from about 1 wt % to about 7 wt %, from about 1 wt % to about 6 wt %, from about 1 wt % to about 5 wt %. In some embodiments, a polypeptide is present in an amount of at least about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or greater than 20 wt %.

In some embodiments, a hydrophilic polymer blend includes a polysaccharide. Such polysaccharides include hydroxypropyl methyl cellulose, carboxymethyl cellulose, methyl ether cellulose, microcrystalline cellulose, chitosan, pullulan, methoxyl pectin, alginic acid (including salts such as sodium alginate, potassium alginate, magnesium alginate, and the like), curdlan, ceral beta glucan, gellan, konjac glucomannan, polydextrose, carrageenan, laminarin, mannan, mannitol, porphyrin, xylan, and salts thereof. In some cases, a polysaccharide is present in an amount of from about 1 wt % to about 50 wt %, from about 1 wt % to about 49 wt %, from about 1 wt % to about 48 wt %, from about 1 wt % to about 47 wt %, from about 1 wt % to about 46 wt %, from about 1 wt % to about 45 wt %, from about 1 wt % to about 44 wt %, from about 1 wt % to about 43 wt %, from about 1 wt % to about 42 wt %, from about 1 wt % to about 41 wt %, from about 1 wt % to about 40 wt %, from about 1 wt % to about 39 wt %, from about 1 wt % to about 38 wt %, from about 1 wt % to about 37 wt %, from about 1 wt % to about 36 wt %, from about 1 wt % to about 35 wt %, from about 1 wt % to about 34 wt %, from about 1 wt % to about 33 wt %, from about 1 wt % to about 32 wt %, from about 1 wt % to about 31 wt %, from about 1 wt % to about 30 wt %, from about 1 wt % to about 29 wt %, from about 1 wt % to about 28 wt %, from about 1 wt % to about 27 wt %, from about 1 wt % to about 26 wt %, from about 1 wt % to about 25 wt %, from about 1 wt % to about 24 wt %, from about 1 wt % to about 23 wt %, from about 1 wt % to about 22 wt %, from about 1 wt % to about 21 wt %, from about 1 wt % to about 20 wt %, from about 1 wt % to about 19 wt %, from about 1 wt % to about 18 wt %, from about 1 wt % to about 17 wt %, from about 1 wt % to about 16 wt %, from about 1 wt % to about 15 wt %, from about 1 wt % to about 14 wt %, from about 1 wt % to about 13 wt %, from about 1 wt % to about 12 wt %, from about 1 wt % to about 11 wt %, from about 1 wt % to about 10 wt %, from about 1 wt % to about 9 wt %, from about 1 wt % to about 8 wt %, from about 1 wt % to about 7 wt %, from about 1 wt % to about 6 wt %, from about 1 wt % to about 5 wt %. In some embodiments, a polysaccharide is present in an amount of at least about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or greater than 20 wt %.

In some embodiments, a hydrophilic polymer blend further includes one or more polyvinyl alcohol polymers. Polyvinyl alcohol polymers include copolymers of vinyl acetate and vinyl alcohol in varying amounts. As such, polyvinyl alcohol polymer can be selected based on the percentage of vinyl alcohol to vinyl acetate monomer (i.e. the “degree of hydrolysis”). In some cases, a polyvinyl alcohol polymer can have a degree of hydrolysis of from about 50% to about 95%, from about 51% to about 95%, from about 52% to about 95%, from about 53% to about 95%, from about 54% to about 95%, from about 55% to about 95%, from about 56% to about 95%, from about 57% to about 95%, from about 58% to about 95%, from about 59% to about 95%, from about 60% to about 95%, from about 61% to about 95%, from about 62% to about 95%, from about 63% to about 95%, from about 64% to about 95%, from about 65% to about 95%, from about 66% to about 95%, from about 67% to about 95%, from about 68% to about 95%, from about 69% to about 95%, from about 70% to about 95%, from about 71% to about 95%, from about 72% to about 95%, from about 73% to about 95%, from about 74% to about 95%, from about 75% to about 95%, from about 76% to about 95%, from about 77% to about 95%, from about 78% to about 95%, from about 79% to about 95%, from about 80% to about 95%, from about 81% to about 95%, from about 82% to about 95%, from about 83% to about 95%, from about 84% to about 95%, from about 85% to about 95%, from about 86% to about 95%, from about 87% to about 95%, from about 88% to about 95%, from about 89% to about 95%, or from about 90% to about 95%. In some specific examples, a degree of hydrolysis can be from about 80% to about 90%. In some cases, a polyvinyl alcohol polymer can include Selvol Ultiloc series 2012, 4005, 5003, and 5103. In some cases, a polyvinyl alcohol polymer is present in an amount of from about 1 wt % to about 50 wt %, from about 1 wt % to about 49 wt %, from about 1 wt % to about 48 wt %, from about 1 wt % to about 47 wt %, from about 1 wt % to about 46 wt %, from about 1 wt % to about 45 wt %, from about 1 wt % to about 44 wt %, from about 1 wt % to about 43 wt %, from about 1 wt % to about 42 wt %, from about 1 wt % to about 41 wt %, from about 1 wt % to about 40 wt %, from about 1 wt % to about 39 wt %, from about 1 wt % to about 38 wt %, from about 1 wt % to about 37 wt %, from about 1 wt % to about 36 wt %, from about 1 wt % to about 35 wt %, from about 1 wt % to about 34 wt %, from about 1 wt % to about 33 wt %, from about 1 wt % to about 32 wt %, from about 1 wt % to about 31 wt %, from about 1 wt % to about 30 wt %, from about 1 wt % to about 29 wt %, from about 1 wt % to about 28 wt %, from about 1 wt % to about 27 wt %, from about 1 wt % to about 26 wt %, from about 1 wt % to about 25 wt %, from about 1 wt % to about 24 wt %, from about 1 wt % to about 23 wt %, from about 1 wt % to about 22 wt %, from about 1 wt % to about 21 wt %, from about 1 wt % to about 20 wt %, from about 1 wt % to about 19 wt %, from about 1 wt % to about 18 wt %, from about 1 wt % to about 17 wt %, from about 1 wt % to about 16 wt %, from about 1 wt % to about 15 wt %, from about 1 wt % to about 14 wt %, from about 1 wt % to about 13 wt %, from about 1 wt % to about 12 wt %, from about 1 wt % to about 11 wt %, from about 1 wt % to about 10 wt %, from about 1 wt % to about 9 wt %, from about 1 wt % to about 8 wt %, from about 1 wt % to about 7 wt %, from about 1 wt % to about 6 wt %, from about 1 wt % to about 5 wt %. In some embodiments, a polyvinyl polymer is present in an amount of at least about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, or greater than about 90 wt %.

In some embodiments, a hydrophilic polymer blend further includes a pyrrolidone polymer such as polyvinylpyrrolidone. A pyrrolidone polymers can further reduce crystallinity of the shelf-stable water-soluble film and are also attractive in the context of cosmetic use as a hair conditioner. In some cases, a pyrrolidone polymer has a molecular weight of from about 20000 g/mol to about 400000 g/mol, from about 30000 g/mol to about 40000 g/mol, from about 40000 g/mol to about 40000 g/mol, from about 50000 g/mol to about 40000 g/mol, from about 60000 g/mol to about 40000 g/mol, from about 70000 g/mol to about 40000 g/mol, from about 80000 g/mol to about 40000 g/mol, from about 90000 g/mol to about 40000 g/mol, from about 100000 g/mol to about 40000 g/mol, from about 110000 g/mol to about 40000 g/mol, from about 120000 g/mol to about 40000 g/mol, from about 130000 g/mol to about 40000 g/mol, from about 140000 g/mol to about 40000 g/mol, from about 150000 g/mol to about 40000 g/mol, from about 160000 g/mol to about 40000 g/mol, from about 170000 g/mol to about 40000 g/mol, from about 180000 g/mol to about 40000 g/mol, from about 190000 g/mol to about 40000 g/mol, from about 200000 g/mol to about 40000 g/mol, from about 210000 g/mol to about 40000 g/mol, from about 220000 g/mol to about 40000 g/mol, from about 230000 g/mol to about 40000 g/mol, from about 240000 g/mol to about 40000 g/mol, from about 250000 g/mol to about 40000 g/mol, from about 260000 g/mol to about 40000 g/mol, from about 270000 g/mol to about 40000 g/mol, from about 280000 g/mol to about 40000 g/mol, from about 290000 g/mol to about 40000 g/mol, from about 300000 g/mol to about 40000 g/mol, from about 310000 g/mol to about 40000 g/mol, from about 320000 g/mol to about 40000 g/mol, from about 330000 g/mol to about 40000 g/mol, from about 340000 g/mol to about 40000 g/mol, from about 350000 g/mol to about 40000 g/mol, from about 360000 g/mol to about 40000 g/mol, from about 370000 g/mol to about 40000 g/mol, from about 380000 g/mol to about 40000 g/mol, or from about 390000 g/mol to about 40000 g/mol. In some cases, a pyrrolidone polymer is present in an amount of from about 1 wt % to about 20 wt %, from about 1 wt % to about 19 wt %, from about 1 wt % to about 18 wt %, from about 1 wt % to about 17 wt %, from about 1 wt % to about 16 wt %, from about 1 wt % to about 15 wt %, from about 1 wt % to about 14 wt %, from about 1 wt % to about 13 wt %, from about 1 wt % to about 12 wt %, from about 1 wt % to about 11 wt %, from about 1 wt % to about 10 wt %, from about 1 wt % to about 9 wt %, from about 1 wt % to about 8 wt %, from about 1 wt % to about 7 wt %, from about 1 wt % to about 6 wt %, or from about 1 wt % to about 5 wt %. In some embodiments, a pyrrolidone polymer is present in an amount of at less than about 20 wt %, 19 wt %, 18 wt %, 17 wt %, 16 wt %, 15 wt %, 14 wt %, 13 wt %, 12 wt %, 11 wt %, 10 wt %, 9 wt %, 8 wt %, 7 wt %, 6 wt %, 5 wt %, 4 wt %, 3 wt %, 2 wt %, 1 wt %.

### Additional Components

A hydrophilic polymer blend as described herein can further comprise additional ingredients. For example, a hydrophilic polymer blend can include bubble-reducing components, plasticizers, or additives such as nucleating agents, surfactants, antiblocking agents, lubricants, biocides, release agents, antioxidants, antifoam, nanoparticles, cross-linking agents, extenders, and fillers.

### Bubble-Reducing Components

As used herein, a “bubble reducing component” includes an ingredient added to the polymer resin blend to reduce bubble formation in the corresponding film, as compared to an otherwise comparable film lacking the bubble reducing component, as determined by heating the film containing the bubble reducing component and the otherwise comparable film and visualizing both after cooling. In some embodiments, a bubble reducing component comprises a polystyrene polymer (e.g. a polystyrene sulfonic acid salt such as sodium polystyrene sulfonate, potassium polystyrene sulfonate, and the like). In some embodiments, a polystyrene sulfonic acid salt is present in an amount of from about 1 wt % to about 10 wt %, from about 1 wt % to about 9 wt %, from about 1 wt % to about 8 wt %, from about 1 wt % to about 7 wt %, from about 1 wt % to about 6 wt %, from about 1 wt % to about 5 wt %, from about 1 wt % to about 4 wt %, or from about 1 wt % to about 3 wt %, with respect to a total weight of the shelf-stable water-soluble film. In some embodiments, a polystyrene sulfonic acid salt has a molecular weight of from about 50,000 g/mol to about 100,000 g/mol (e.g. about 70,000 g/mol).

In some embodiments, a shelf-stable water-soluble film comprises alginic acid or a salt thereof. Examples of alginate salts include sodium alginate, magnesium alginate, calcium alginate, and the like. In some instances, the alginic acid or salt thereof can be present in an amount sufficient to reduce bubble formation in the film as described above. In some instances, the alginic acid or salt thereof are part of the hydrophilic polymer blend.

In some embodiments, a shelf-stable water-soluble film comprises iso-tridecyl alcohol. In some instances, the iso-tridecyl alcohol can be present in an amount sufficient to reduce bubble formation in the film as described above.

### Plasticizers

In some cases, a hydrophilic polymer blend further comprises an amount of a plasticizer. Plasticizers offer a further reduction in crystallinity in the polymeric resin. In some cases, a mixture of plasticizers of both high molecular weight and small molecular weight materials can be added. Without wishing to be bound by theory, high molecular weight plasticizers reduce the large crystalline regions in the film, while the small molecular weight plasticizers reduce the small crystalline regions.

Polymeric plasticizers are polymers which lack the molecular weight to grant strong film forming attributes, however, may grant a further reduction in crystallinity. Some embodiments include polymeric plasticizers because materials with a larger molecular weight find permanence in the polymer resin. Examples of such materials include polyvinyl alcohol polymers as described above, with some embodiments including PVA with a degree of hydrolysis between 80 and 88% and with a molecular weight less than 15,000 g/mol (e.g. less than 10,000 g/mol). Similarly, polyvinylpyrrolidone may be included as described above, with embodiments including PVP with a molecular weight less than 15,000 g/mol (e.g. less than 10,000 g/mol). A polyoxazoline as described above such as poly(2-ethyl-2-oxazoline) with a molecular weight below 5,000 Daltons may be incorporated as well. Additionally, embodiments may include polyether polyols such as polyethylene glycol, polypropylene oxide, polybutylene oxide with molecular weight up to 1000 g/mol. Similarly, polyoxypropylene and/or polyethylene glycol ethers may be included. In some embodiments, one or more polymeric plasticizers may be included in a range of from about 0.1 wt % to about 25 wt %, relative to the total mass of the hydrophilic polymer resin.

Non-polymeric plasticizers which are solid at room temperature may be included as well. In some embodiments, such materials have a dipole moment greater than 2, (e.g. greater than 3). Without wishing to be bound by theory, some materials with a high dipole moment may not partition towards a liquid hygiene product as described herein. Examples of such materials include amides such as carbamide and sugar alcohols such as sorbitol. Such materials may be included in a range of from about 0.1 wt % to about 15 wt %, relative to the total mass of the hydrophilic polymer resin.

Non-polymeric plasticizers which are liquid at room temperature may be included as well. Triol ethoxylates, such as glycereth-26 and glycereth-7, and methyl glucoside ethoxylates, such as methyl gluceth-10 and methyl gluceth-20, are included in some embodiments. In some embodiments, one or more non-polymeric plasticizers are included in a range of from about 0.1 wt % to about 15 wt %, relative to the total mass of the hydrophilic polymer resin. Other non-limiting examples include amino alcohols such as triethanolamine, and low molecular weight polyhydric alcohols.

As discussed above, incorporation of low molecular weight polyhydric alcohols is limited in some embodiments provided herein. A “low molecular weight polyhydric alcohol” as used herein refers to those with a molecular weight below 200 g/mol. Low molecular weight polyhydric alcohols, such as propylene glycol and glycerin, can promote various desirable properties such as very effective plasticizing, compatibilizers, and increasing the heat-sealing temperature range for thermal-processability. Where present, a small molecular weight polyhydric alcohol is present in an amount of from about 0.5 wt % to about 5 wt %, from about 0.5 wt % to about 4 wt %, from about 0.5 wt % to about 3 wt %, from about 0.5 wt % to about 2 wt %, or from about 0.5 wt % to about 1 wt %, with respect to a total weight of the shelf-stable water-soluble film.

A hydrophilic polymer resin can also comprise water content as discussed above. Water can also act as a plasticizer in the resulting film. The resulting water content may be controlled by processing conditions, but more commonly by controlling the weight fraction of humectants in the film. In some embodiments, water content can be measured by Karl Fischer titration. In some cases, a film's water content is measured after allowing the film to equilibrate for 72 hours at 74+/− Fahrenheit at 50% RH. In some cases, water content can be at least about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % with respect to a total weight of the shelf-stable water-soluble film. In some cases, a film's water content is measured at 40 degrees Celsius at 80% RH. In some cases, a film's water content is measured at 40 degrees Celsius at 10% RH. In some cases, the water soluble film is exposed to these conditions for up to 42 days. In some bases, the water soluble film is evaluated every 14 days under the conditions described herein. In some cases a film's water content is measured to accelerate conditions detecting an incompatibility between a water soluble film, and an anhydrous cosmetic composition.

Control of the total amount of plasticizer can affect the properties of the resulting film. Without wishing to be bound by theory, if too much plasticizing material is present, then the plasticizers will partition from the film's matrix as the polymer resin achieves a thermodynamically favored state, whereas if too little plasticizing material is present, then the resulting film will lack the proper elasticity to be processed. Accordingly, in some embodiments the total amount of non-film forming plasticizing material ranges from about 10 wt % to about 40 wt %, from about 20 wt % to about 30 wt %, or from about 20 wt % to about 25 wt %, relative to the total mass of the hydrophilic polymer resin.

### Additives

As discussed above, a hydrophilic polymer blend can further include additives. In some embodiments, an additive includes a nucleating agent. Without wishing to be bound by theory, Nucleating agents establish an ionic lattice upon which the polymeric resin may establish itself. This can, in some cases, promote superior resin crystallization and modify barrier properties. Nucleating agents include materials like inorganic salts such as sodium sulfate and magnesium chloride, inorganic silicate salts such as magnesium silicate, and naturally occurring siliceous such as diatomaceous earth. For such materials, a particle size of from about 1 to about 25 microns (e.g. from about 1 to about 10 microns) can be used. If the material is too large, then the likelihood of pinhole formation during thermoforming increase. If the material is too small, then the likelihood of achieving a homogeneous dispersion decrease. In some embodiments, a nucleating agent where added in present in an amount of from about 0.5 wt % to about 15 wt % relative to the total mass of the film forming polymer resin.

In some embodiments, an additive includes a surfactant. Without wishing to be bound by theory, surfactants promote a homogeneous dispersion, support ease of removal from cast surface, and additionally may plasticize some crystalline regions. A range of anionic, non-ionic, and/or amphoteric surfactants may be used. Examples include carboxylic acid salts, phosphate esters, alkylphenol ethoxylates, alcohol ethoxylates, ester ethoxylates, alkyl polyglucosides, alkanolamides, ethylene oxide—propylene oxide co polymers, polysorbate 20, polysorbate 60, polysorbate 65, polysorbate 80, amine oxides, and amphoaceates. In some specific embodiments, a non-ionic surfactant is added. In some embodiments, a surfactant when added is present in an amount of from about 0.1 wt % to about 2.0 wt % relative to the total mass of the film forming polymer resin.

In some embodiments, an additive is an anti-blocking agent. Without wishing to be bound by theory, use of anti-blocking agents may reduce the coefficient of friction on the film's surface. Consequently, reducing the likelihood of the film adhering to itself or to a surface. Furthermore, it may reduce the likelihood of adhering to skin type surfaces during handling. Examples include inorganic salts, microcrystalline cellulose, saturated fatty acids salts, silica derivatives, layered silica derivatives, and clay minerals. In some embodiments, anti-blocking agents when added are present in an amount of from about 0.5 wt % to about 8 wt % relative to the total mass of the film forming polymer resin.

In some embodiments, an additive is a lubricant. Without wishing to be bound by theory, lubricants migrate to the film: air interface and modify surface properties such as coefficient of friction and water droplet contact angle. Examples of suitable materials include fatty acids, fatty alcohols, fatty esters, fatty amines, fatty amine acetylates, and fatty amides. In some embodiments, a lubricant when added is present in an amount of from about 0.5 wt % to about 8 wt %, relative to the total mass of the film forming polymer resin.

In some embodiments, an additive is a biocide. In some embodiments, a suitable biocide when added is present in an amount of from about 0.1 wt % to about 2 wt % final dry mass weight. Suitable biocides include quaternary ammonium salts, halogenated salicylanilides, and bithionol, among various others.

Additional additives include, but is not limited to, release agents, antioxidants, antifoam, nanoparticles, cross-linking agents, extenders, and fillers.

### Methods of Making Water-Soluble Films

Also disclosed herein are methods of making water-soluble films as described herein. In some embodiments, a water-soluble film can be prepared by first dissolving a hydrophilic polymer blend in water, followed by heating the hydrophilic polymer blend to generate a polymer blend film. The mixture is then cooled. Additional additives as described above can be added before or after dissolving the hydrophilic polymer blend.

Alternatively, the hydrophilic polymer blend may be dispersed and solubilized using a high shear mixer and/or homogenizer such as those manufactured by Silverson. Using such a process, advantageously removes the need for a heat cycle, effectively introducing cost and time savings during manufacturing. Expectedly, the additives may be dispersed and solubilized as well.

The film may be produced by solvent casting. In a laboratory setting, the film may be cast by hand using a doctor's blade. The resulting cast slurry may be left to dry overnight in an under ambient pressure. Alternatively, the cast slurry may be dried in a vacuum oven up to 70 Celsius and 700 mg Hg vacuum pressure. Further, the polymer slurry may be continuously cast by solvent cast film manufacturing equipment.

The resulting film thickness may be measured by a digital micrometer. In most embodiments, the resulting film may have a thickness of from about 2 mil to about 4 mil (e.g. from about 2 mil to about 3 mil). Without being bound to any particular theory, a thicker film may provide superior barrier properties against the passive diffusion of the encapsulated contents.

### Performance

A shelf-stable water-soluble film comprising a hydrophilic polymer blend and other ingredients as described herein provides superior properties relative to comparable films. For example, a shelf-stable water-soluble film of the present disclosure has reduced crystallinity, and as such has improved water solubility relative to comparable films. Further, the resulting polymer blend film can form a miscible blend of polymer components. This may be concluded by observing a single glass transition temperature as measured by differential scan calorimetry or dynamic mechanical analysis. In some embodiments, the resulting film has a homogeneous appearance. In some embodiments, the film dissolves uniformly and tears perpendicular to an applied stress.

In some embodiments, a shelf-stable water-soluble film of the present disclosure at a thickness of about 3 mil when contacted with water at 26° C. has a disintegration time of less than about 60 seconds (e.g. less than about 59 seconds, less than about 58 seconds, less than about 57 seconds, less than about 56 seconds, less than about 55 seconds, less than about 54 seconds, less than about 53 seconds, less than about 52 seconds, less than about 51 seconds, less than about 50 seconds, less than about 49 seconds, less than about 48 seconds, less than about 47 seconds, less than about 46 seconds, less than about 45 seconds, less than about 44 seconds, less than about 43 seconds, less than about 42 seconds, less than about 41 seconds, less than about 40 seconds, less than about 39 seconds, less than about 38 seconds, less than about 37 seconds, less than about 36 seconds, less than about 35 seconds, less than about 34 seconds, less than about 33 seconds, less than about 32 seconds, less than about 31 seconds, less than about 30 seconds, less than about 29 seconds, less than about 28 seconds, less than about 27 seconds, less than about 26 seconds, less than about 25 seconds, less than about 24 seconds, less than about 23 seconds, less than about 22 seconds, less than about 21 seconds, less than about 20 seconds, less than about 19 seconds, less than about 18 seconds, less than about 17 seconds, less than about 16 seconds, less than about 15 seconds, less than about 14 seconds, less than about 13 seconds, less than about 12 seconds, less than about 11 seconds, less than about 10 seconds).

In some embodiments, a shelf-stable water-soluble film of the present disclosure at a thickness of about 3 mil when contacted with water at 26° C. has a dissolution time of less than about 60 seconds (e.g. less than about 59 seconds, less than about 58 seconds, less than about 57 seconds, less than about 56 seconds, less than about 55 seconds, less than about 54 seconds, less than about 53 seconds, less than about 52 seconds, less than about 51 seconds, less than about 50 seconds, less than about 49 seconds, less than about 48 seconds, less than about 47 seconds, less than about 46 seconds, less than about 45 seconds, less than about 44 seconds, less than about 43 seconds, less than about 42 seconds, less than about 41 seconds, less than about 40 seconds, less than about 39 seconds, less than about 38 seconds, less than about 37 seconds, less than about 36 seconds, less than about 35 seconds, less than about 34 seconds, less than about 33 seconds, less than about 32 seconds, less than about 31 seconds, less than about 30 seconds, less than about 29 seconds, less than about 28 seconds, less than about 27 seconds, less than about 26 seconds, less than about 25 seconds, less than about 24 seconds, less than about 23 seconds, less than about 22 seconds, less than about 21 seconds, less than about 20 seconds, less than about 19 seconds, less than about 18 seconds, less than about 17 seconds, less than about 16 seconds, less than about 15 seconds, less than about 14 seconds, less than about 13 seconds, less than about 12 seconds, less than about 11 seconds, less than about 10 seconds).

In some embodiments, a water-soluble film as described herein has increased tensile strength. The stress strain response is measured by ASTM D882 “Standard Test Method for Tensile Properties of Thin Plastic Sheeting”. The test may be conducted on a universal tensile tester such as a Texture Analyzer XT.TC plus C with tensile grip attachments. In some embodiments, the stress at film rupture is from about 10 Mpa to about 30 Mpa, from about 15 Mpa to about 30 Mpa, from about 20 Mpa to about 30 Mpa, or from about 250 Mpa to about 30 Mpa. In some embodiments, the stress at film rupture is greater than about 14 MPa (e.g. greater than about 18 Mpa, greater than about 20 MPa). Similarly, the elongation at break should be greater than 450% (e.g. greater than 500%, greater than 600%).

In some embodiments, a water-soluble film resists aging over a given time period. Without wishing to be bound by theory, as the film ages it is observed that the plasticizers leach out from the film. This may occur because the film naturally reverts to a thermodynamically favored crystalline state over time. However, the process may be controlled with the proper selection of ingredients as described herein. In some embodiments, film compositions display no signs of plasticizer migration after a time period of at least 1 month, at least 3 months, and or at least 6 months of aging at 74+/−4 Fahrenheit at 50% RH. Leaching of plasticizers may be observed by swiping a black nitrile glove over the surface of the film; if plasticizer material is leaching, then a visible streak should be visible on the nitrile glove.

### Hygiene Product Nods

Also disclosed herein are hygiene product pods made from a shelf-stable water-soluble film containing a hygiene product as described herein. A hygiene product can be encapsulated into the shelf-stable water-soluble film in the form of a water-soluble envelope.

Various embodiments described herein may include a single dose of a hygiene product in the form of a pod. As used herein, the term “pod” refers to water-soluble material filled with a hygiene product and sealed to form a chamber (also referred to herein as an envelope) in the shape of a capsule, sphere, drop, or the like. For example, a pod may include a water-soluble envelope encapsulating a liquid or gel hygiene product. In some embodiments, a pod may include a water-soluble envelope encapsulating a solid or a paste hygiene product.

A pod may have any suitable shape and/or size as desired by one of ordinary skill in the art. For example, the water-soluble material may be sealed around the hygiene product to form a shaped chamber holding the hygiene product. In some embodiments, the chamber may be relatively flat (e.g., with a relatively small thickness compared to its width and length) such that the pod appears nearly two-dimensional. In some embodiments, the chamber may be three-dimensional. The chamber (and thus the pod as a whole) may be shaped like a capsule, a sphere, a drop, a cylinder, a cone, an ellipsoid, a tetrahedron, a square pyramid, a hexagonal pyramid, a cube, a cuboid, a triangular prism, an octahedron, a pentagonal prism, a hexagonal prism, a dodecahedron, an icosahedron, or the like, or a nearly two-dimensional version (e.g., rectangular, circular, triangular, square, pentagonal, circular, elliptical, tablet-shaped, or the like) thereof.

In some embodiments, a pod may contain a sufficient amount of hygiene product for a single use (also referred to herein as a single dose). For example, a pod may contain an amount of shampoo sufficient to clean one head of hair. In some embodiments, a pod may contain an amount of conditioner sufficient to condition one head of hair. In some embodiments, a pod may contain an amount of bodywash sufficient to wash one body. In some embodiments, a pod may contain an amount of a shaving product sufficient to shave one or more body parts. As used herein, the term “shaving product” may refer to shaving cream, shaving gel, or a precursor composition configured to form a shaving product. It will be understood by one of ordinary skill in the art that the volume of a single dose of the hygiene product may depend on the nature of the hygiene product itself. For example, a user may require a smaller volume of a more concentrated hygiene product and a larger volume of a more dilute hygiene product in order to achieve the same effect. Alternatively, or in combination, the type of hygiene product may determine the volume of a single dose thereof. For example, a single dose of a shampoo may be about the same as a single dose of a conditioner. A single dose of a bodywash may be larger than a single dose of a shampoo if the bodywash is less viscous than the shampoo and/or if the tissue surface area to be cleansed is sufficiently larger. Pods including a concentrated active agent may be smaller than pods containing undiluted or semi-diluted active agents.

The pod may have a weight within a range of about 1 g to about 20 g. For example, the pod may have a weight within a range bounded by any two of the following values: 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, or 20 g. In some embodiments, for example when the hygiene product is a shampoo, conditioner, or shampoo and conditioner two-in-one, the pod may range in weight from about 4 g to about 12 g, such as from about 5 g to about 8 g. In some embodiments, for example when the hygiene product is a bodywash, the pod may range in weight from about 5 g to about 15 g, such as from about 8 g to about 10 g.

The pod may have a volume within a range of about 4 ml to about 10 ml. For example, the pod may have a volume within a range bounded by any two of the following values: 4 ml, 5 ml, 6 ml, 7 ml, 8 ml, 9 ml, or 10 ml. In some embodiments, for example when the hygiene product is a shampoo or bodywash, the pod may range in volume from about 4 ml to about 7 ml, such as about 5 ml. In some embodiments, for example when the hygiene product is a conditioner, the pod may range in volume from about 6 ml to about 8 ml, such as about 7 ml.

FIG. 1A shows a photograph of a plurality of hygiene product pods 100. The pod 100 comprises a hygiene product 104 sealed in a water-soluble envelope 102. The water-soluble envelope 102 is sealed around the hygiene product 104 to form a hygiene product 104 filled chamber. The chamber (and thus the overall impression of the pod 100) may be generally drop shaped (as shown). However, the chamber/pod is not limited to any particular shape. For example, the chamber/pod may be rectangular, circular, triangular, square, pentagonal, circular, elliptical, tablet-shaped, or the like.

The water-soluble envelope 102 may have a thickness ranging from about 0.5 mil to about 200 mil, from about 0.5 mil to about 190 mil, from about 0.5 mil to about 180 mil, from about 0.5 mil to about 170 mil, from about 0.5 mil to about 160 mil, from about 0.5 mil to about 150 mil, from about 0.5 mil to about 140 mil, from about 0.5 mil to about 130 mil, from about 0.5 mil to about 120 mil, from about 0.5 mil to about 110 mil, from about 0.5 mil to about 100 mil, from about 0.5 mil to about 90 mil, from about 0.5 mil to about 80 mil, from about 0.5 mil to about 70 mil, from about 0.5 mil to about 60 mil, from about 0.5 mil to about 50 mil, from about 0.5 mil to about 40 mil, from about 0.5 mil to about 30 mil, from about 0.5 mil to about 20 mil, from about 0.5 mil to about 10 mil, from about 0.5 mil to about 9 mil, from about 0.5 mil to about 8 mil, from about 0.5 mil to about 7 mil, from about 0.5 mil to about 6 mil, from about 0.5 mil to about 5 mil, from about 0.5 mil to about 4 mil, from about 0.5 mil to about 3 mil, from about 0.5 mil to about 2 mil, or from about 0.5 mil to about 1 mil. For example, the water-soluble envelope 102 may have a thickness within a range bounded by any two of the following values: 0.5 mil, 1 mil, 1.5 mil, 2 mil, 2.5 mil, 3 mil, 3.5 mil, 4 mil, 4.5 mil, 5 mil, 5.5 mil, 6 mil, 6.5 mil, 7 mil, 7.5 mil, 8 mil, 8.5 mil, 9 mil, 9.5 mil, 10 mil, 11 mil, 12 mil, 13 mil, 14 mil, 15 mil, 16 mil, 17 mil, 18 mil, 19 mil, 20 mil, 25 mil, 30 mil, 35 mil, 40 mil, 45 mil, 50 mil, 55 mil, 60 mil, 65 mil, 70 mil, 75 mil, 80 mil, 85 mil, 90 mil, 95 mil, 100 mil, 105 mil, 110 mil, 115 mil, 120 mil, 125 mil, 130 mil, 135 mil, 140 mil, 145 mil, 150 mil, 155 mil, 160 mil, 165 mil, 170 mil, 175 mil, 180 mil, 185 mil, 190 mil, 195 mil, or 200 mil.

The water-soluble envelope 102 may have a pH ranging from about 4.0 to about 9.0, at a temperature of about 25° C. For example, the water-soluble envelope 102 may have a pH at about 25° C. within a range bounded by any two of the following values: 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

In various embodiments, sealing of a hygiene product within a water-soluble envelope described herein may be accomplished using any suitable sealing method. In some embodiments, the water-soluble material may be sealed using a heat sealing method. In some embodiments, sealing may include the use of a water-soluble adhesive. In some embodiments, hygiene product pods 100 may be formed using a packing machine, such as a Hydroforma packaging machine manufactured by Cloud Packaging Solutions, Des Plaines, IL.

FIG. 1B shows a perspective front view of a hygiene product pod 100 following an optional trimming process.

FIG. 1C shows a side view of the hygiene product pod 100 of FIG. 1B. In some embodiments, at least a portion of the seal region 106 may be optionally removed from the hygiene product pod 100, for example to form a tab 108. For example, the water-soluble envelope 102 may be cut during manufacturing of the hygiene product pod 100. The tab 108 may operate as a contact point to facilitate handling of the hygiene product pod 100 and/or removal of the hygiene product pod 100 from product container or packaging.

FIG. 1D shows a perspective front view of a hygiene product pod 100. FIG. 1E shows a top view of the hygiene product pod of FIG. 1D. As described herein, the hygiene product pod 100 may comprise a hygiene product 104 sealed in a water-soluble envelope 102. The water-soluble envelope 102 may be sealed around the hygiene product 104 to form a hygiene product 104 filled chamber. In some embodiments, at least a portion of the seal region 106 may be optionally removed from the hygiene product pod 100, for example to form tabs 108. In some embodiments, the chamber (and thus the overall impression of the pod 100) may be generally spherical-shaped or planetoid shaped (as shown). The hygiene product pod 100 may comprise one or more tabs 108, for example two tabs 108 on opposite ends of the seal region 106.

FIG. 1F shows a perspective front view of a hygiene product pod 100.

FIG. 1G shows a top view of the hygiene product pod 100 of FIG. 1F. As described herein, the hygiene product pod 100 may comprise a hygiene product 104 sealed in a water-soluble envelope 102. The water-soluble envelope 102 may be sealed around the hygiene product 104 to form a hygiene product 104 filled chamber. In some embodiments, at least a portion of the seal region 106 may be optionally removed from the hygiene product pod 100, for example to form tab 108. In some embodiments, the chamber (and thus the overall impression of the pod 100) may be generally spherical-shaped (as shown).

FIG. 1H shows a perspective front view of a hygiene product pod 100. As described herein, the hygiene product pod 100 may comprise a hygiene product 104 sealed in a water-soluble envelope 102. The water-soluble envelope 102 may be sealed around the hygiene product 104 to form a hygiene product 104 filled chamber. In some embodiments, at least a portion of the seal region 106 may be optionally removed from the hygiene product pod 100, for example to form tab 108. In some embodiments, the chamber (and thus the overall impression of the pod 100) may be generally drop-shaped (as shown).

FIG. 1I shows a perspective front view of a hygiene product pod 100. As described herein, the hygiene product pod 100 may comprise a hygiene product 104 sealed in a water-soluble envelope 102. The water-soluble envelope 102 may be sealed around the hygiene product 104 to form a hygiene product 104 filled chamber. In some embodiments, at least a portion of the seal region 106 may be optionally removed from the hygiene product pod 100, for example to form tab 108. In some embodiments, the chamber may be generally spherical-shaped (as shown) and the tab 108 may be tapered such that the overall impression of the pod 100 is roughly drop shaped.

FIG. 1J shows a perspective front view of a hygiene product pod 100. As described herein, the hygiene product pod 100 may comprise a hygiene product 104 sealed in a water-soluble envelope 102. The water-soluble envelope 102 may be sealed 106 around the hygiene product 104 to form a hygiene product 104 filled chamber. In some embodiments, the chamber (and thus the overall impression of the pod 100) may be generally square-shaped (as shown).

FIG. 2 shows a top plan view of a hygiene product pod 110. The hygiene product pod 110 may be substantially similar to the pod 100, so only the differences therebetween will be described in detail. The pod 110 may comprise a water-soluble envelope 102 in which a hygiene product 104 is disposed as described herein. In contrast to the pod 100, the water-soluble envelope 102 may be formed by sealing a single folded sheet of the water-soluble material around the hygiene product 104. As a result, the hygiene product pod 110 may include a seal region 106 that extends along three sides of the hygiene product 104 and a folded region 112 that extends between opposing ends of the seal region 106.

FIG. 3 shows a top view of a hygiene product pod 120. The hygiene product pod 120 may be similar to the hygiene product pod 100, so only the differences therebetween will be described in detail. The pod 120 may comprise a water-soluble envelope 102 in which a hygiene product 104 is disposed as described herein. In contrast to the hygiene product pod 100, the water-soluble envelope 102 may be formed by sealing opposing ends of a tubular sheet of the water-soluble material following deposition of the hygiene product 104 therein. As a result, the hygiene product pod 120 may include seal regions 106 disposed at opposing ends of the hygiene product pod 120.

### Hygiene Products

As disclosed above, a shelf-stable water-soluble film can encapsulate a hygiene product to produce a hygiene product pod. The present disclosure is described in relation to hygiene products (including shampoo, conditioner, bodywash, soap, shaving cream, lotion, etc.). However, one of skill in the art will appreciate that this is not intended to be limiting and the devices and methods disclosed herein may be used in other cleaning and/or personal care products. For example, the devices and methods disclosed herein may be used to provide single-use water soluble laundry detergents, bleach, laundry additives (e.g., softener), fabric care products, dishwashing detergents, surface cleaning products, beauty care products, skin care products (e.g., lotions), toothpastes, etc.

According to various embodiments, the present hygiene product may have a free water content of about 18% or less, such as about 15% wt % or less, about 12 wt % or less, about 9 wt % or less, or about 5 wt % or less. For example, the hygiene product may have a free water content ranging from about 1 wt % to about 12.5 wt %, about 1 wt % to about 9 wt %, from about 1 wt % to about 7 wt %, or from about 1 wt % to about 5 wt %. The hygiene product may have a free water content within a range bounded by any two of the following values: 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, or 18 wt %. The free water content may be determined using methods and systems that will be understood by one of ordinary skill in the art based on the teachings herein. For example, the free water content may be measured using a Karl Fischer volumetric titrator.

In some embodiments, the hygiene product may have a water activity of 0.70 a_{w} or less in order to maintain product stability. For example, the hygiene product may have a water activity within a range bounded by any two of the following values: 0.10 a_{w}, 0.20 a_{w}, 0.30 a_{w}, 0.40 a_{w}, 0.50 a_{w}, 0.60 a_{w}, or 0.70 a_{w}. Water activity may be measured using methods and systems that will be understood by one of ordinary skill in the art based on the teachings herein. For example, the water activity of the hygiene product may be measured with a resistive electrolytic hygrometer, a capacitance hygrometer, a dew point hygrometer, or by equilibration, or the like.

In some embodiments, the hygiene product may be in the form of a substantially non-aqueous liquid or gel cleansing product, such as a shampoo, a conditioner, a bodywash, or any combination thereof, such as a combined shampoo, bodywash, and/or conditioner. In some embodiments, the hygiene product may be in the form of a substantially non-aqueous shaving product, such as a shaving gel, cream, liquid, or the like.

The hygiene product may include one or more active agents dispersed in a multifunctional carrier. Additional thickeners and other secondary ingredients may also be added to the hygiene product. The one or more active agents may include one or more surfactants (e.g., a surfactant system comprising a mixture of surfactants), one or more cleansing agents, and/or one or more conditioning agents. When present, the one or more surfactants and/or surfactant system may comprise one or more primary surfactants which, when combined with water and optionally mechanically agitated (e.g., rubbed), generate a foam or a froth. When present, the conditioning agents may be configured to counteract the inherent harshness of the surfactants. The conditioning agents, multifunctional carrier, thickeners (e.g., gellants), and/or secondary ingredients may be configured to increase the integrity of a hygiene product pod and/or may operate to disperse the active agent when the hygiene product pod is dissolved in water.

In various embodiments, the active agents may be in the form of a concentrate, such as a shampoo concentrate, a conditioner concentrate, a bodywash concentrate, or any combinations thereof. The active agents may be in the form of a shaving product concentrate in other embodiments. In some embodiments, the active agent may include one or more of the above components in a powdered format.

The multifunctional carrier may be non-aqueous. The multifunctional carrier may be configured such that the hygiene product has rheological properties that are expected by consumers. The multifunctional carrier may also facilitate encapsulation of the hygiene product in a water-soluble envelope and/or may operate to maintain the long-term stability of the hygiene product and/or the water-soluble envelope.

In various embodiments, the multifunctional carrier may operate as surfactant vehicle and/or as a solvent. In some embodiments, the multifunctional carrier may act as a secondary surfactant and enhance the bubble-forming properties of the primary surfactant. In some embodiments, the multifunctional carrier may be selected or configured to lower the overall reactivity of the hygiene product and the water-soluble envelope. For example, the multifunctional carrier may operate to control the free water percentage of the hygiene product.

In some embodiments, the multifunctional carrier may be a non-aqueous liquid or gel. The multifunctional carrier may be configured to moisten one or more of the hygiene product pod components. In some embodiments, the multifunctional carrier may also operate as a conditioner for hair and/or skin. In some embodiments, the multifunctional carrier may comprise one or more secondary surfactants or cosurfactants. The multifunctional carrier may include, for example, polyols, polyglyceryls, phospholipids, liquid amides, or combinations thereof.

In some embodiments, the hygiene product may comprise one or more primary surfactants. In some embodiments, the hygiene product may not comprise a primary surfactant. For example, a shampoo or bodywash hygiene product may comprise one or more primary surfactants while a conditioner or lotion hygiene product may comprise little (e.g., 5% or less) to no primary surfactants.

### Primary Surfactants

In various embodiments, the active agents of the hygiene product may include one or more cleansing agents, such as surfactants. For example, the hygiene product may include one or more anionic, nonionic, cationic, and/or amphoteric surfactants. In some embodiments, the hygiene product may comprise a surfactant system including a combination of different surfactants. The following surfactants may be referred to herein as “primary surfactants”.

Non-limiting examples of suitable anionic surfactants include alkali metal sulforicinates, sulfonated glyceryl esters of fatty acids, such as sulfonated monoglycerides of coconut oil acids, salts of sulfonated monovalent alcohol esters such as sodium oleylisethianate, metal soaps of fatty acids, amides of amino sulfonic acids such as the sodium salt of oleyl methyl tauride, sulfonated products of fatty acids nitriles, such as palmitonitrile sulfonate, sulfonated aromatic hydrocarbons such as sodium alpha-naphthalene monosulfonate, condensation products of naphthalene sulfonic acids with formaldehyde, sodium octahydroanthracene sulfonate, alkali metal alkyl sulfates such as sodium lauryl sulfate, ammonium lauryl sulfate or triethanolamine lauryl sulfate, ether sulfates having alkyl groups of 8 or more carbon atoms, such as sodium lauryl ether sulfate, ammonium lauryl ether sulfate, sodium alkyl aryl ether sulfates, and ammonium alkyl aryl ether sulfates, alkylarylsulfonates having one or more alkyl groups of 8 or more carbon atoms, alkylbenzenesulfonic acid alkali metal salts exemplified by hexylbenzenesulfonic acid sodium salt, octylbenzenesulfonic acid sodium salt, calcium salts, decylbenzenesulfonic acid sodium salt, dodecylbenzenesulfonic acid sodium salt, cetylbenzenesulfonic acid sodium salt, and myristylbenzenesulfonic acid sodium salt, sulfuric esters of polyoxyethylene alkyl ether including CH₃(CH₂)₆CH₂O(C₂H₄O)₂SO₃H, CH₃(CH₂)₇CH₂O(C₂H₄O)_{3.5}SO₃H, CH₃(CH₂)₈CH₂O(C₂H₄O)₈SO₃H, CH₃(CH₂)₁₉CH₂O(C₂H₄O)₄SO₃H, and CH₃(CH₂)₁₀CH₂O(C₂H₄O)₆SO₃H, sodium salts, potassium salts, and amine salts of alkylnapthylsulfonic acid.

Non-limiting examples of suitable cationic surfactants include various fatty acid amines and amides and their derivatives, and the salts of the fatty acid amines and amides. Examples of aliphatic fatty acid amines include dodecylamine acetate, octadecylamine acetate, and acetates of the amines of tallow fatty acids, homologues of aromatic amines having fatty acids such as dodecylanalin, fatty amides derived from aliphatic diamines such as undecylimidazoline, fatty amides derived from aliphatic diamines, such as undecylimidazoline, fatty amides derived from disubstituted amines such as oleylaminodiethylamine, derivatives of ethylene diamine, quaternary ammonium compounds and their salts which are exemplified by tallow trimethyl ammonium chloride, dioctadecyldimethyl ammonium chloride, didodecyldimethyl ammonium chloride, dihexadecyl ammonium chloride, alkyltrimethylammonium hydroxides such as octyltrimethylammonium hydroxide, dodecyltrimethylammonium hydroxide, or hexadecyltrimethylammonium hydroxide, dialkyldimethylammonium hydroxides such as octyldimethylammonium hydroxide, decyldimethylammonium hydroxide, didodecyldimethylammonium hydroxide, dioctadecyldimethylammonium hydroxide, tallow trimethylammonium hydroxide, trimethylammonium hydroxide, methylpolyoxyethylene cocoammonium chloride, and dipalmityl hydroxyethylammonium methosulfate, amide derivatives of amino alcohols such as beta-hydroxylethylstearylamide, and amine salts of long chain fatty acids.

Non-limiting examples of suitable cationic surfactants include also quaternary ammonium halides such as octyl trimethyl ammonium chloride, dodecyl trimethyl ammonium chloride, hexadecyl trimethyl ammonium chloride, octyl dimethyl benzyl ammonium chloride, decyl dimethyl benzyl ammonium chloride and coco trimethyl ammonium chloride as well as other salts of these materials, fatty amines and basic pyridinium compounds, quaternary ammonium bases of benzimidazolines, polypropanolpolyethanol amines, polyethoxylated quaternary ammonium salts and ethylene oxide condensation products of the primary fatty amines, available from Armak Company, Chicago, Ill. under the tradenames Ethoquad, Ethomeen, or Arquad. Suitable cationic surfactants can also be an esterquat-type compound.

In some embodiments, stearamidopropyl dimethylamine is a cationic, flaked, solid tertiary amine surfactant that may be included in the hygiene product. Stearamidopropyl dimethylamine is an effective conditioning agent and emulsifier. Stearamidopropyl dimethylamine also improves wet combing and feel, lowers dry hair friction, and softens hair.

Non-limiting examples of suitable nonionic surfactants include capryloyl/caproyl methyl glucamide and lauroyl/myristoyl methyl glucamide, lauryldimethylamine oxide (e.g., lauramine oxide), decyl glucosides, polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters. Suitable nonionic surfactants include condensates of ethylene oxide with a long chain (fatty) alcohol or (fatty) acid, condensates of ethylene oxide with an amine or an amide, condensation products of ethylene and propylene oxides, fatty acid alkylol amide and fatty amine oxides. Examples of non-ionic surfactants include polyoxyalkylene alkyl ethers such as polyethylene glycol long chain (12-14C) alkyl ether, polyoxyalkylene sorbitan ethers, polyoxyalkylene alkoxylate esters, polyoxyalkylene alkylphenol ethers, ethylene glycol propylene glycol copolymers, polyvinyl alcohol, and alkylpolysaccharides.

Non-limiting examples of suitable amphoteric surfactants include aliphatic secondary or tertiary amine derivatives in which the aliphatic radical is a linear or branched chain containing 8 to 22 carbon atoms and containing-at least one water-soluble anionic group (for example carboxylate, sulphonate, sulphate, phosphate or phosphonate); mention may also be made of (C₈-C₂₀)alkyl-betaines, sulphobetaines, (C₈-C₂₀)alkylamido(C₁-C₆)alkyl-betaines, or (C₈-C₂₀)alkylamido(C₁-C₆)alkylsulphobetaines. In some embodiments, bocamidopropyl betaine (CAPB) may be included in the hygiene product as an amphoteric surfactant.

In some embodiments, environmentally friendly surfactants may be used, such as sulfate-free surfactants, such as sodium lauryl sulfoacetate, alpha olefin sulfonate, or the like, or combinations thereof.

In some embodiments, the hygiene product may include surfactants derived from natural oils, such as coconut oil, safflower oil, or the like, or natural materials such as sugar. For example, the hygiene product may include a sodium cocoyl isethionate (Hostapon SCI-85 C, manufactured by Clariant Corp.), cocamidopropyl betaine (Chembetaine C-42, manufactured by Lubrizol Corp.), cocamidopropyl hydroxysultaine (Chembetaine CAS manufactured by Lubrizol Corp.), capryloyl/caproyl methyl glucamide and lauroyl/myristoyl methyl glucamide (GlucoTain Plus, manufactured by Clariant Corp.), sodium methyl 2-sulfolaurate (Alpha Step PC-48, manufactured by Stepan Co.), alkyl polyglucoside (Pantaren 2000, manufactured by BASF Corp.) lauramine oxide (Mackamine LO, manufactured by Solvay Novecare Corp.), sodium methyl cocoyl taurate, or combinations thereof.

In various embodiments, the hygiene product may include one or more primary surfactants, such as one or more nonionic surfactants, one or more anionic surfactants, one or more cationic surfactants, and/or one or more amphoteric surfactants, or any combination thereof. For example, the hygiene product may include one or more primary surfactants in an amount ranging from about from about 0 wt % to about 50 wt %, such as from about 5 wt % to about 50 wt %, about 10 wt % to about 40 wt %, from about 20 wt % to about 35 wt %, from about 16 wt % to about 34 wt %, or from about 17 to about 33 wt %. In some embodiments, the one or more primary surfactants may be within a range of about 0 wt % to about 5 wt % or within a range of about 10 wt % to about 50 wt %. For example, a conditioner or lotion hygiene product may include the one or more primary surfactants in an amount ranging from about 0 wt % to about 5 wt % while a shampoo or bodywash hygiene product may include the one or more primary surfactants in an amount ranging from about 20 wt % to about 30 wt %. In various embodiments, the hygiene product may include one or more primary surfactants in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 20 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, or 50 wt %. The amount of primary surfactant(s) in the hygiene product may vary depending on the type of hygiene product (e.g., conditioner, lotion, shampoo, shaving product, etc.) and the desired lather of the hygiene product.

In various embodiments, the hygiene product may include a mixture of primary surfactants, such as nonionic surfactants or a combination of nonionic and anionic surfactants or any combination of primary surfactants desired. In some embodiments, the hygiene product may include a surfactant mixture in an amount ranging from about 0 wt % to about 50 wt %, such as from about 5 wt % to about 50 wt %, about 10 wt % to about 40 wt %, from about 20 wt % to about 35 wt %, from about 16 wt % to about 34 wt %, or from about 17 to about 33 wt %. %. In some embodiments, the surfactant mixture may be within a range of about 0 wt % to about 5 wt % or within a range of about 10 wt % to about 50 wt %. For example, a conditioner or lotion hygiene product may include the surfactant mixture in an amount ranging from about 0 wt % to about 5 wt % while a shampoo or bodywash hygiene product may include the surfactant mixture in an amount ranging from about 20 wt % to about 30 wt %. In various embodiments, the hygiene product may include a surfactant mixture in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 20 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, or 50 wt %.

In some embodiments, such as in shampoo and/or bodywash applications, the surfactant system may be free of cationic surfactants. In other words, cationic surfactants may be excluded from the hygiene product in certain applications.

In some embodiments, the surfactant system may include at least one nonionic surfactant in an amount ranging from about 7 wt % to about 25 wt %, such as from about 8 wt % to about 22 wt %, or from about 9 wt % to about 20 wt %. In various embodiments, the surfactant system may include at least one nonionic surfactant in an amount within a range bounded by any two of the following values: 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt %.

In some embodiments, the surfactant system may include at least one anionic surfactant in an amount ranging from about 0 wt % to about 25 wt %, such as from about 8 wt % to about 22 wt %, or from about 9 wt % to about 20 wt %. In various embodiments, the surfactant system may include at least one anionic surfactant in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt %.

In some embodiments, the surfactant system may have an anionic surfactant to nonionic surfactant weight ratio ranging from about 0 to about 2, such as from about 0.5 to about 1.9, or from about 1.3 to about 1.4. In various embodiments, the surfactant system may have an anionic surfactant to nonionic surfactant weight ratio within a range bounded by any two of the following values: 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2.

In some embodiments, the surfactant system may include surfactants selected from sodium cocoyl isothionate, sodium methyl cocoyl taurate, cocamidopropyl hydroxysultaine, capryloyl/caproyl methyl glucamide and lauroyl/myristoyl methyl glucamide, sodium methyl 2-sulfolaurate, alkyl polyglucoside, or any combinations thereof.

According to various embodiments of the present disclosure, the hygiene product may include one or more liquid amides. The liquid amides may be configured to operate as primary or secondary surfactants, solubilizers, emollients, and/or emulsifiers.

For example, the hygiene product may include one or more liquid amides in an amount ranging from about 0 wt % to about 45 wt %, such as from about 0 wt % to about 15 wt %, from about 5 wt % to about 15 wt %, from about 5 wt % to about 40 wt %, from about 6 wt % to about 20 wt %, or from about 8 wt % to about 15 wt %. In various embodiments, the hygiene product may include one or more liquid amides in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 20 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, or 45 wt %.

The one or more liquid amides may include Glucotain Plus, chembetaines, lactamide monoethanolamine (LMEA), Dimethyl lauramide/myristamide, or the like, or any combination thereof. In some embodiments, one or more liquid amide may be a primary surfactant. Alternatively, or in combination, one or more liquid amide may be a secondary surfactant, solubilizer, emollient, emulsifier, and/or diluent.

For example, lactamide monoethanolamine (LMEA) (Phoenamid LMEA, available from Phoenix Chemical, Inc.) is a liquid amide humectant that may impart smooth, non-greasy feel and sheen to skin and/or hair.

In various embodiments, the hygiene product may include LMEA in an amount ranging from about 0 wt % to about 20 wt %, such as from about 1 wt % to about 15 wt %, from about 2 wt % to about 12 wt %, or from about 3 wt % to about 9 wt %. In various embodiments, the surfactant system may include LMEA in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt %.

Dimethyl lauramide/myristamide (Ninol CAA, available from Stephan Co.) is a liquid amide secondary surfactant configured to optimize carrier characteristics without reducing cleansing performance. Ninol CAA is a nitrosamine-free, 100% active, liquid amide that enables the imperceptible reduction of surfactant levels, solubilizes fragrances, and may substitute other amides.

In various embodiments, the hygiene product may include Ninol CAA in an amount ranging from about 0 wt % to about 20 wt %, such as from about 1 wt % to about 15 wt %, from about 3 wt % to about 10 wt %, or from about 2 wt % to about 4 wt %. In various embodiments, the surfactant system may include Ninol CAA in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt %.

### Polyol Solvents

In some embodiments, the multifunctional carrier may include one or more polyol solvents that are compatible with shelf-stable water-soluble film described herein. For example, the one or more polyol solvents may include nonvolatile, low molecular weight molecules. Accordingly, one or more of the polyol solvents may function as a plasticizer to improve the flexibility and processability of the water-soluble envelope.

The one or more polyol solvents may operate as solvents, humectants, and/or may control viscosity. In some embodiments, the hygiene product may include from about 5 wt % to about 75 wt %, such as from about 10 wt % to about 70 wt %, about 20 wt % to about 72 wt %, about 40 wt % to about 75 wt %, about 5 wt % to about 35 wt %, about 10 wt % to about 30 wt %, from about 15 wt % to about 30 wt %, from about 15 wt % to about 25 wt %, of the one or more polyol solvents. In various embodiments, the hygiene product may include one or more polyol solvents in an amount within a range bounded by any two of the following values: 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 20 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, or 75 wt %.

In some embodiments, the hygiene product may include a mixture of two or more polyol solvents. For example, the hygiene product may include from about 5 wt % to about 75 wt %, such as from about 10 wt % to about 70 wt %, about 20 wt % to about 72 wt %, about 40 wt % to about 75 wt %, about 5 wt % to about 35 wt %, about 10 wt % to about 30 wt %, from about 15 wt % to about 25 wt %, or about 20 wt %, of the polyol solvent mixture. In various embodiments, the hygiene product may include polyol solvent mixture in an amount within a range bounded by any two of the following values: 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, or 75 wt %.

In some embodiments, the polyols may include glycols and/or glycerin. For example, the glycols may be selected from ethylene glycol, poly(ethylene glycol), butylene glycol (e.g., 1,3-butylene glycol), hexylene glycol, propylene glycol, or dipropylene glycol. In some embodiments, the polyol mixture may include of at least two glycols, at least three glycols, or at least four glycols, and may additionally include glycerin.

Hexylene glycol may provide anti-microbial activity, emollience, moisturization, and/or skin conditioning effects to the hygiene product. Hexylene glycol may also operate to plasticize the water-soluble envelope.

According to various embodiments, the hygiene product may include from 0 wt % to about 5 wt %, such as from about 0.5 wt % to about 4 wt %, or from about 1 wt % to about 3 wt % hexylene glycol. In some embodiments, the hexylene glycol content may be limited to about 2 wt %, in order to reduce eye irritation. In some embodiments, the hexylene glycol content may be within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, or 5 wt %.

Propylene glycol is highly hydrophilic and may operate as a humectant that moisturizes the skin and/or hair. Propylene glycol may also operate as a solvent to the active agents and may operate as a plasticizer with respect to the water-soluble envelope.

According to various embodiments, the hygiene product may include, based on the total weight of the hygiene product, from about 5 to about 25 wt %, such as from about 10 wt % to about 20 wt %, or about 15 wt % propylene glycol. In some embodiments, the polyol mixture may include propylene glycol primary component. In other words, the polyol solvent mixture may include more propylene glycol than other polyol solvents. In various embodiments, the hygiene product may include propylene glycol in an amount within a range bounded by any two of the following values: 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, or 30 wt %.

Glycerin operates as an active ingredient solvent, an envelope plasticizer, and a humectant. According to various embodiments, the hygiene product may include from about 0.5 wt % to about 15 wt %, such as from about 1 wt % to about 10 wt %, or from about 2 wt % to about 8 wt % glycerin. In some embodiments, the glycerin content may be limited to 8 wt % or less, to reduce and/or prevent degradation to the water-soluble envelope. In some embodiments, the glycerin content may be within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, or 15 wt %.

Di-propylene glycol is a highly effective solvent, solubilizing agent, and carrying agent for fragrances. The hygiene product may include an amount of di-propylene glycol sufficient to solubilize an amount of fragrance oils and/or essential oils included in the hygiene product. For example, the hygiene product may include a 2:1 weight ratio of di-propylene glycol to fragrances and/or essential oils.

According to various embodiments, the hygiene product may include from 0 wt % to about 7 wt %, such as from about 0.1 wt % to about 6 wt %, or about 2 wt % to about 5 wt % di-propylene glycol. In some embodiments, the di-propylene glycol content may be within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, or 7 wt %.

Butylene glycol operates as a humectant that moisturizes the skin and/or hair. Butylene glycol may also operate as a solvent with respect to the active agents, and may operate as a plasticizer with respect to the water-soluble envelope. Butylene glycol may also operate as a conditioning agent.

According to various embodiments, the hygiene product may include, based on the total weight of the hygiene product, from 0 wt % to about 10 wt %, such as from about 0.5 wt % to about 8 wt %, or from about 2 wt % to about 6 wt %, butylene glycol. In some embodiments, the butylene glycol content may be limited to 10 wt % or less, in order to prevent an excessive viscosity increase in the hygiene product. In some embodiments, the hygiene product may be free of butylene glycol. In some embodiments, the butylene glycol content may be within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, or 10 wt %.

### Polyglyceryl Fatty Acid Ester Chassis

In some embodiments, the multifunctional carrier may include one or more polyglyceryl fatty acid ester (PGE) chassis configured to, for example, provide emulsification and/or increase foaming. The PGE chassis may operate to solvate the active agents without reducing envelope stability. In some instances, the PGE chassis may act as solubilizers, rheology modifiers, diluents, water-replacement ingredients in a near-anhydrous system, and/or non-reactive carrier solvents. The PGE chassis may provide body to the hygiene product without significantly affecting the structure and/or solubility of the water-soluble envelope.

The PGE chassis may include polyglyceryl monoesters and multi-esters. In shampoo and bodywash applications, the PGE chassis may have a hydrophilic-lipophilic balance (HLB) value of at least 9, such as at least 10 or at least 11. For example, the PGE chassis may comprise polyglyceryl-2 caprate, polyglyceryl-3 caprate, polyglyceryl-2 laurate, polyglyceryl-3 laurate, polyglyceryl-6 esters, polyglyceryl-2 oleate, polyglyceryl-2 monostearate, or any combinations thereof.

In various embodiments, the hygiene product may include from about 0 wt % to about 60 wt %, such as from about 0.1 to about 60 wt %, from about 0 wt % to about 50 wt %, from about 10 wt % to about 50 wt %, from about 12 wt % to about 49 wt %, or from about 15 wt % to about 35 wt % of the PGE chassis. For example, the PGE chassis may be about 30% for a shampoo or conditioner hygiene product and about 50% for a bodywash hygiene product. In various embodiments, the hygiene product may include the PGE chassis in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, or 60 wt %.

Polyglyceryl-2 caprate is a liquid compound derived from polyglycerin and a fatty acid. Polyglyceryl-2 caprate may operate as an emulsifier to create fine, creamy bubbles, especially when combined with anionic surfactants. It is an excellent thickener with good thermostable viscosity. Polyglyceryl-2 caprate can be used as deodorizing ingredient because of its reaction to gram-positive bacteria, making it a desirable addition for anhydrous body wash and shampoo formulations.

The hygiene product may include, from about 0 wt % to about 50 wt %, such as from about 0.1 wt % to about 40 wt %, or from about 5 wt % to about 30 wt %, polyglyceryl-2 caprate. Amounts of over 50 wt % are possible, but may prevent the hygiene product from including appropriate amounts of other ingredients, such as surfactants, conditioning agents, etc., for a given application. In various embodiments, the hygiene product may include polyglyceryl-2 caprate in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, or 50 wt %.

Polyglyceryl-3 caprate is a liquid nonionic co-surfactant and refatting agent with an estimated HLB value of 10-13. This polyethylene glycol (PEG)-free refatting agent helps to prevent the skin from drying and provides a luxurious and soft skin feel. It also provides a mild antimicrobial and deodorizing effect.

The hygiene product may include from about 0 wt % to about 25 wt %, such as from about 0.1 wt % to about 20 wt %, or from about 5 wt % to about 15 wt %, polyglyceryl-3 caprate. It is believed that polyglyceryl-3 caprate amounts of greater than 25% may result in decreased foam height and bubble size in some formulations. In various embodiments, the hygiene product may include polyglyceryl-3 caprate in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt %.

In some embodiments, the hygiene product may include polyglyceryl-2 caprate esters in an amount ranging from about 10 wt % to about 30 wt %, and polyglyceryl-3 caprate in an amount ranging from about 4 wt % to about 24 wt %.

Polyglyceryl-3 laurate may provide moisturization, humectant, and emollient properties in both hair and skin formulations. Polyglyceryl-3 laurate is also completely dispersible in water.

The hygiene product may include from about 0 wt % to about 30 wt %, such as from about 0.1 wt % to about 20 wt %, or from about 5 wt % to about 18 wt %, polyglyceryl-3 laurate. It is believed that polyglyceryl-3 laurate amounts of greater than 20 wt % may result in decreased foam height and bubble size in some formulations. In various embodiments, the hygiene product may include polyglyceryl-3 laurate in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, or 30 wt %.

Sunflower oil polyglyceryl-6 esters may be obtained by the transesterification of sunflower seed oil and polyglycerin-6. The compound is an amphiphilic ester that promotes shine and skin and hair softening attributes without negatively affecting the foam by contributing to emollient overload.

The hygiene product may include from about 0 to about 15 wt %, such as from about 0.1 wt % to about 12 wt %, from about 1 wt % to about 11 wt %, or from about 2 wt % to about 7 wt %, polyglyceryl-6 esters. It is believed that the hygiene product may include polyglyceryl-6 esters in amounts of up to 50 wt %. However, amounts of 15 wt % or less may be preferable due to the extreme cost and low commercial availability of this ingredient. In some embodiments, the hygiene product may include polyglyceryl-6 esters in an amount within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 20 wt %, 25 wt %, 30 wt %, 35 wt %, 40 wt %, 45 wt %, or 50 wt %.

In some embodiments, the hygiene product may include polyglyceryl-6 esters in an amount ranging from about 1 wt % to about 11 wt %, and polyglyceryl-2 caprate in an amount ranging from about 5 wt % to about 15 wt %.

In conditioning applications, the hygiene product may include PGEs having HLB values of about 9 or less, such as about 8 or less, or about 6 or less. In such hygiene products, the PGEs may be used in conjunction with cationic conditioning agents and triglycerides.

### Additional Conditioners

In some embodiments, the hygiene product may include one or more additional conditioners. Non-limiting examples of suitable additional conditioners include petrolatum, fatty acids, esters of fatty acids, fatty alcohols, ethoxylated alcohols, polyol polyesters, glycerin monoesters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, synthetic waxes, polyolefinic glycols, polyolefinic monoester, polyolefinic polyesters, cholesterols, cholesterol esters, triglycerides and mixtures thereof.

Preferable additional conditioners may include pyrrolidone carboxylic acid (PCA) glyceryl Oleate (SOFTISAN conditionHAIR, available from IOI Oleo GmbH), which is a natural skin and hair conditioning agent, caprylyl pyrrolidone, lauryl pyrrolidone, betaine (Genencare OSMS BA, available from DuPont Nutrition and Biosciences Inc.), polyethylene glycol/fumed silica (POLYOX, available from DuPont Nutrition and Biosciences Inc.), and polyethylene oxide (molecular weight within a range of about 100,000 to about 600,000).

An additional conditioner as described herein can be added in an amount of from about 0 to about 15 wt %, such as from about 0.1 wt % to about 12 wt %, from about 0.5 wt % to about 10 wt %, or from about 1 wt % to about 5 wt %. In some embodiments, the additional condition can be present at a weight percent of: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, or 20 wt %.

### Additional Emulsifiers Stabilizers

In various embodiments, the hygiene product may include one or more additional emulsifiers that may operate to further stabilize the hygiene product. For example, the hygiene product may include non-ionic, oil-in-water emulsifiers configured to create a lamellar or bilayered structure. For example, the emulsifiers may include non-ionic emulsifiers, such as, a mixture of cetearyl alcohol and cetearyl glucoside (Montanov 68, available from Seppic Corp.), glycerol stearate (Lipo GMS 450, available from Lipo Chemicals Corp.), or a combination thereof.

In some embodiments, the hygiene product may include a non-ionic, oil-in-water emulsifier in an amount ranging from about 0 wt % to about 3 wt %, such as from about 0.2 wt % to about 2 wt %, or from about 0.8 wt % to about 1.3 wt %. In various embodiments, hygiene product may include a non-ionic, oil-in-water emulsifier in an amount within a range bounded by any two of the following values: 0 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, or 3 wt %.

In some embodiments, the emulsifiers may include polyethylene glycol/fumed silica (POLYOX, available from DuPont Nutrition and Biosciences Inc.), polyethylene glycol, hygroscopic polymers, such as guar gum (e.g., cyamopsis tetragonoloba gum), cationic guar gum, xanthan gum, starch, pregelatinized starch, modified starches, such as hydroxypropyl starch phosphate and sodium starch glycolate, honey, polyvinyl pyrrolidone (PVP), ExpertGel EG312 (poloxamer 338/PPG-12/SMDI copolymer) available from DKSH Inc., ExpertGel EG412 (poloxamer 407, PPG-12/SMDI copolymer) available from DKSH Inc, combinations thereof, or the like. In some embodiments, the thickeners may include cellulose derivatives such as carboxymethyl cellulose, cellulose gum, or tylose powder. In some embodiments, the hygiene product may include a polyoxyethylene ether.

In some embodiments, the emulsifiers may include a hydroscopic polymer thickener in an amount ranging from about 0 wt % to about 0.4 wt %, such as from about 0.05 wt % to about 0.3 wt %, or from about 0.1 wt % to about 0.2 wt %. In various embodiments, hygiene product may include a hydroscopic polymer thickener in an amount within a range bounded by any two of the following values: 0 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.11 wt %, 0.12 wt %, 0.13 wt %, 0.14 wt %, 0.15 wt %, 0.16 wt %, 0.17 wt %, 0.18 wt %, 0.19 wt %, 0.2 wt %, 0.21 wt %, 0.22 wt %, 0.23 wt %, 0.24 wt %, 0.25 wt %, 0.26 wt %, 0.27 wt %, 0.28 wt %, 0.29 wt %, 0.3 wt %, 0.31 wt %, 0.32 wt %, 0.33 wt %, 0.34 wt %, 0.35 wt %, 0.36 wt %, 0.37 wt %, 0.38 wt %, 0.39 wt %, 0.4 wt %.

For example, the hygiene product may include a modified starch thickener in an amount ranging from about 0.1 wt % to about 3 wt %, such as from about 0.2 wt % to about 2 wt %, or from about 0.3 wt % to about 1 wt %. In various embodiments, hygiene product may include a modified starch thickener in an amount within a range bounded by any two of the following values: 0 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, or 3 wt %.

In some embodiments, the emulsifiers may include nonionic surfactants configured to form a lamellar network. For example, the emulsifiers may include a lamellar network formed from polyoxyethylene ethers including a mixture of high molecular mass saturated fatty alcohols, mainly cetyl alcohol and stearyl alcohol, such as ceteareth-20. In some embodiments, the hygiene product may include a nonionic surfactant in an amount ranging from about 8 wt % to about 18 wt %, such as about 12 wt %.

Lauryl lactyl lactate (Stepan-Mild L3, available from Stephan Co.) is a naturally derived, multi-functional emollient/surfactant formed as the product of reacting lauryl alcohol and lactic acid. Stepan-Mild L3 may operate as a viscosity builder, foam booster, emulsifier, and emollient.

In various embodiments, the hygiene product may include STEPAN-MILD L3 in an amount ranging from about 0 wt % to about 20 wt %, such as from about 0.5 wt % to about 15 wt %, from about 1 wt % to about 10 wt %, or from about 2 wt % to about 7 wt %. In some embodiments, the Stepan-Mild L3 content may be within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, or 20 wt %.

According to various embodiments of the present disclosure, the hygiene product may include one or more phospholipids configured to operate as a foam enhancer and/or cleanser. For example, the hygiene product may include from about 0 wt % to about 20 wt %, such as from about 1 wt % to about 15 wt %, from about 2 wt % to about 15 wt %, from about 5 wt % to about 10 wt %, or from about 6 wt % to about 8 wt % of the one or more phospholipids. In some embodiments, the one or more phospholipids may be within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt %, 11 wt %, 11.5 wt %, 12 wt %, 12.5 wt %, 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, or 20 wt %.

In some embodiments, the hygiene product may include phospholipids selected from cocamidopropyl PG-dimonium chloride phosphate (Cola Lipid C, manufactured by Colonial Chemical Inc.), linoleamidopropyl PG-dimonium chloride phosphate (Cola Lipid SAFL, manufactured by Colonial Chemical Inc.), or combinations thereof.

In some embodiments, the hygiene product may include substantially equal amounts of different phospholipids. For example, the hygiene product may include substantially equal amounts of Cola Lipid C and Cola Lipid SAFL.

### Carrier Diffusion Control

In some embodiments, the water-soluble envelope of the hygiene product pod may have some amount of permeability with respect to the hygiene product. This could lead to diffusion of one or more components of the hygiene product through the water-soluble envelope over time, which may reduce pod shelf-life. For example, a water-soluble envelope formed using a shelf-stable water-soluble film as described herein could inherently have some amount permeability with respect the butylene glycol carrier. Accordingly, the hygiene product may include thickeners configured to reduce carrier permeation through the water-soluble envelope.

In various embodiments, thickeners such as carboxymethyl cellulose, cellulose gum, or tylose powder may be optionally added to the hygiene product to reduce diffusion through the water-soluble envelope. For example, tylose powder may be added to the hygiene product in an amount ranging from about 0.05 wt % to about 0.5 wt %, such as from about 0.15 wt % to about 0.25 wt %, or about based on the total weight of the hygiene product.

### Secondary Ingredients

In various embodiments, the hygiene product pods may include secondary ingredients. For example, the secondary ingredients may include buffers/pH adjusters, dyes/colorants, moisturizers, fragrances, vitamins, texture modifiers, essential oils, foam enhancers, and anti-microbial agents, combinations thereof, or the like. In various embodiments, hygiene products may include from about 0 wt % to about 8 wt % secondary ingredients. In some embodiments, the secondary ingredients may be within a range bounded by any two of the following values: 0 wt %, 0.5 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, or 8 wt %.

The buffers/pH adjusters may include calcium ions, potassium ions, or hydroxide ions, any combination thereof, or any salts or compounds capable of generation such ions. The buffers/pH adjusters may be blended at various ratios, in order to provide a suitable pH. For example, a suitable amount of buffer/pH adjuster may be added to the hygiene product to provide a slightly acidic pH for compatibility with water-soluble envelopes. For example, the hygiene product may advantageously have a room temperature pH ranging from about 5 to about 9, such as from about 4.8 to about 6.9, or from about 5.0 to about 6.8. In some embodiments, the hygiene product may have a room temperature pH within a range bounded by any two of the following values: 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9.

In some embodiments, the secondary ingredients may include essential oils such as argan oil, sunflower oil, lavender oil, rosemary oil, cedar wood oil, thyme oil, peppermint oil, chamomile oil, sage oil, lemon oil, patchouli oil, tea tree oil, ylang oil, vetiver oil, carrot seed oil, cypress oil, helichrysum oil, combinations thereof, or the like.

In some embodiments, hygiene products may include moisturizers such as betaine (Genencare OSMS BA, manufactured by Dupont Corp.), polyquaterium-10, or brassica alcohol and brassicyl valinate esylate (AminoSensyl™ HC). In some embodiments, hygiene products may include vitamins such as tocopherol, DL Panthenol, or combinations thereof.

Secondary ingredients useful for improving dissolvability and/or foaming may include coconut milk powder, arrow root powder, colloidal oatmeal powder, combinations thereof, or the like. In various embodiments, hygiene products may include a secondary ingredient, such as coconut milk powder, arrow root powder, colloidal oatmeal powder, or any combination thereof, in an amount ranging from about 0 wt % to about 22 wt %, such as from about 5 wt % to about 20 wt %, or from about 8 wt % to about 15 wt %. Colloidal oatmeal powder may also operate as a texture modifier to provide a smoother texture. In various embodiments, the hygiene product may include a secondary ingredient, such as coconut milk powder, arrow root powder, colloidal oatmeal powder, or any combination thereof, in an amount within a range bounded by any two of the following values: 0 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, or 22 wt %.

Any suitable colorant may be used. Hygiene products may include a colorant in an amount ranging from about 0 wt % to about 0.3 wt %, such as from about 0.1 wt % to about 0.2 wt %.

In various embodiments, a suitable fragrance may be used. Useful fragrances may be in liquid form, such as traditional fragrances that are combinations of synthetic and natural compounds, natural fragrances that consist of a blend of natural extracts and essential oils, or essential oils in the pure and neat form. Hygiene products may include a fragrance oil in an amount ranging from about 0 wt % to about 1.5 wt %, such as from about 0.5 wt % to about 1 wt %. In various embodiments, hygiene product may include a fragrance oil in an amount within a range bounded by any two of the following values: 0 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, or 1.5 wt %.

In various embodiments, texture modifiers may include micro or macro abrasive agents, such as when a pod includes a body scrub. Suitable abrasive agents include, for example, nut powders, silica powders, polymer beads such as wax beads, combinations thereof, or the like. In some embodiments, hygiene products may include an abrasive agent in an amount ranging from about 0 wt % to about 3 wt %, such as from about 0.1 wt % to about 2 wt %, or from about 0.2 wt % to about 1 wt %. In various embodiments, hygiene product may include an abrasive agent in an amount within a range bounded by any two of the following values: 0 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, or 3 wt %.

In various embodiments, anti-microbial agents may be natural materials having anti-microbial effects. For example, anti-microbial agents may include thyme oil, tea tree oil, oregano oil, lavender oil, citrus essential oil, grapefruit seed extract, olive leaf extract, honey, or the like. Hygiene products may include an anti-microbial agent in an amount ranging from about 0 wt % to about 3 wt %, such as from about 0.1 wt % to about 2 wt %, or from about 0.2 wt % to about 1 wt %. In various embodiments, hygiene product may include an anti-microbial agent in an amount within a range bounded by any two of the following values: 0 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 2.1 wt %, 2.2 wt %, 2.3 wt %, 2.4 wt %, 2.5 wt %, 2.6 wt %, 2.7 wt %, 2.8 wt %, 2.9 wt %, or 3 wt %.

### Compositions

Also disclosed herein are compositions that comprise a water-soluble film as described herein.

### Active Ingredients

In some embodiments, a composition can comprise a water-soluble film as described herein and an active ingredient. For example, a composition can comprise a water-soluble film and a drug, a medicament, a pharmaceutical, a nutraceutical, a supplement, or a combination thereof. In some cases, the composition comprises the water-soluble film encapsulating the active ingredient (e.g. a drug, a medicament, a pharmaceutical, a nutraceutical, a supplement, or a combination thereof). In some cases, the composition comprises the water-soluble film coated with the active ingredient (e.g. a drug, a medicament, a pharmaceutical, a nutraceutical, a supplement, or a combination thereof). In some cases, the composition comprises the water-soluble film as a component of a container or packaging containing the active ingredient (e.g. a drug, a medicament, a pharmaceutical, a nutraceutical, a supplement, or a combination thereof). For example, the water-soluble film can be a component of a capsule or unit dosage form that can deliver the active ingredient. In some cases, the water-soluble film can be in the form of a slip (e.g. a soap slip) containing an active ingredient described herein embedded therein.

In some cases, the active ingredient can be a moisturizer, a vitamin (e.g. vitamin E, vitamin C, vitamin B3), an oil (e.g. argan oil, avocado oil, tea tree oil, olive oil, castor oil, sunflower oil, lavender oil, rosemary oil, cedar wood oil, jojoba oil, thyme oil, peppermint oil, chamomile oil, sage oil, lemon oil, patchouli oil, tea tree oil, ylang oil, acai oil, vetiver oil, carrot seed oil, cypress oil, helichrysum oil, hemp seed oil, rosehip oil, and combinations thereof), retinoic acid, retinol, hyaluronic acid, an alpha-hydroxy acid, a poly hydroxy acid, a beta-hydroxy acid, ceramide, green tea extract, an algae extract, salicylic acid, linoleic acid, azelaic acid, kaolin clay, CoQ10, lactic acid, centella asiatica, niacinamide, caffeine, glycolic acid, licorice, benzoyl peroxide, glutathione, bakuchiol, mandelic acid, squalene, snail gel, arbutin, aloe vera, honey, biotin, seaweed, tapioca, charcoal, shea butter, cocoa butter, and combinations thereof.

### Cleaning Agents

In some embodiments, the composition can comprise a water-soluble film as described herein and a cleaning agent. In some cases, the composition comprises the water-soluble film as a component of a container or packaging containing the cleaning agent. In some cases, a cleaning agent can be a household care product. A household care product can include, for example, light duty liquid detergents compositions, heavy duty liquid detergent compositions, hard surface cleaning compositions, laundry detergent gels, bleaching compositions, laundry additives, or fabric enhancer compositions. A household care product as described herein can comprise a nonionic alkoxylated surfactant (e.g, ethoxylates derived from C6-C18 primary alcohols), an ethoxylated-propoxylated alcohol, an epoxy-capped poly(oxyalkylated) alcohol, an ether-capped poly(oxyalkylated) alcohol surfactant, a block polyoxyethylene-polyoxypropylene polymeric compound; an amphoteric surfactant such as the C12-C20 alkyl amine oxides (e.g. amine oxides such as lauryldimethyl amine oxide and hexadecyl dimethyl amine oxide), an alkyl amphocarboxylic surfactant; a zwitterionic surfactants such as a betaine or sultaine; and mixtures thereof. In some cases, a household care product can comprise enzymes such as hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. In some cases, a household care product can comprise bleaching agents such as sodium percarbonate, sodium hypochlorite, or tetraacetyl ethylene diamine. In some cases, a household care product can comprise glycerol, propylene glycol, isopropanol, Marlipal C₁₂₋₁₄EO₇, C₁₃₋₁₅EO₉, C₉₋₁₁EO₉), alhylbenzene, alkylbenzenesulfonate, alkylsulfate, dimethyl hydroxyethylammonium chloride, MEA-LAS, MEA-Laureth Sulfate, PEI ethoxylate, C₁₂₋₁₆ Pareth, C₁₄₋₁₅ Pareth, C₁₀₋₁₆ Pareth, propylene glycol, acrylamide, bentonite, benzisothiozolinone, C₁₀₋₁₆ alkyldimethylamine oxide, ethanolamine citrate, ethanolamine laurate, ethanolamine myristate, ethanolamine palmitate, ethanolamine oleate, ethanolamine stearate, palmitic acid, tripropylene glycol, and combinations thereof.

### Methods of Assessing Compatibility

As discussed above, a liquid hygiene product should be compatible with a shelf-stable water-soluble film (i.e. the liquid hygiene product should not phase separate when encapsulated in the water-soluble envelope, and should not dissolve the water-soluble envelope when encapsulated). The term “compatibility” refers to the interchange of components between a film and what the film encapsulates. If there is a lot of interchange, the solubility of the film degrades over time.

Compatibility can be measure by placing a water-soluble envelope containing a hygiene product inside at varying conditions and measuring performance as described above. For example, the product pod can be stored at temperatures ranging from 25° C. to 50° C. (e.g. about 30° C., about 34° C., about 38° C., about 40° C.) and under relative humidity ranging from about 20% to about 80%. In some cases, compatibility of a water-soluble envelope with a liquid hygiene product described herein satisfies the following as analyzed on day 42 after storage under conditions described above (e.g. about 38° C. and 80% relative humidity) of compatibility testing:

$$Water content of cosmetic formulation < 25 \%$$

$$Force to burst film > 1200 g$$

$$Distance to burst < 20 mm$$

$$Disintegration time < 25 seconds$$

$$Dissolution time < 45 seconds$$

Furthermore, a compatible envelope generally successfully contains the encapsulated contents, with little to no diffusion of liquid hygiene product across the film membrane. Additional compatibility criteria include a dry touch and lack of catalyzed phase separation.

### EXAMPLES

For a belier understanding of the present disclosure and of its many advantages, the following examples are given by way of illustration and without limiting the scope of this disclosure.

### Example 1—Hygiene Product Compositions

Tables 1 and 2 include example formulations 1-15 of exemplary hygiene product compositions for use with water-soluble envelopes described herein.

TABLE 1
Exemplary hygiene product compositions 1-7

| | Ex. 1 | Ex. 2 | Ex. 3 | Ex. 4 | Ex. 5 | Ex. 6 | Ex. 7 |
|---|---|---|---|---|---|---|---|
| Component | Wt. % | Wt. % | Wt. % | Wt. % | Wt. % | Wt. % | Wt. % |
| Hostapon SCI-85 P | 13 | 14.3 | | 11 | | 8 | 10.5 |
| Chembetaine CAS | 3 | 3 | 3 | 3 | 3 | 3 | 3 |
| Glucotain Plus | 6 | 9 | 6 | 6 | 6 | 5 | 8.5 |
| Alpha Step PC-48 | | | | | | | 6 |
| Alkyl | | | 8 | 11 | 11 | 2 | 1 |
| Polyglucoside | | | | | | | |
| Propylene Glycol | 5 | 9 | 9 | 9 | 11 | 11.5 | 14 |
| Hexylene Glycol | 2 | 2 | 2 | 2 | 2 | 2 | 2 |
| Di-propylene | 7 | 6 | 6 | 5 | 6 | 4 | 2 |
| Glycol | | | | | | | |
| Glycerin | 3 | 4 | 4 | 4 | 4 | 4 | 6 |
| Polyglyceryl-6 Est. | | | | 10 | 10 | 7 | 3 |
| Polyglyceryl-3 Cap. | | | | | | | |
| Polyglyceryl-2 Cap. | | | 12.5 | 10.5 | 10.9 | 10 | 14 |
| Cola Lipid C | 5 | 4 | 4 | 4 | 4 | 4 | 4 |
| Cola Lipid SAFL | 3 | 2 | 2 | 2 | 2 | 2 | 2 |
| Phoenamid LMEA | 11 | 11 | 11 | 8 | 9 | 9.1 | 7.8 |
| Stepan Mild L3 | 10.85 | 12 | 13 | 2 | 4 | 4 | 5 |
| Nino CAA | 10.85 | 11 | 12 | 2 | 4 | 4 | 3 |
| Additives | 19.2 | 9.4 | 7.5 | 10 | 13.1 | 11.4 | 6.6 |

TABLE 2
Exemplary hygiene product compositions 8-15

| | Ex. 8 | Ex. 9 | Ex. 10 | Ex. 11 | Ex. 12 | Ex. 13 | Ex. 14 | Ex. 15 |
|---|---|---|---|---|---|---|---|---|
| Component | Wt. % | Wt. % | Wt. % | Wt. % | Wt. % | Wt. % | Wt. % | Wt. % |
| Hostapon SCI-85 P | 11.5 | 9 | 13 | 8 | 11 | 13 | 10 | 11.5 |
| Chembetaine CAS | 3 | 3 | 3 | 3 | 3 | 3 | 3 | 3 |
| Glucotain Plus | 8.5 | 5.5 | 5.5 | 5.5 | 5.5 | 5.5 | 5.5 | 5.5 |
| Alpha Step PC-48 | 6 | 3.5 | 4.5 | 4.5 | 5.5 | 5.5 | 5 | 4.35 |
| Alkyl | 1 | 1 | 1 | 1 | 1 | 1 | 1.2 | 1.5 |
| Polyglucoside | | | | | | | | |
| Propylene Glycol | 18.5 | 14 | 14 | 5 | 15 | 15 | 14 | 14 |
| Hexylene Glycol | 2 | 2 | 2 | 2 | 2 | 2 | 2 | 2 |
| Di-propylene | | | | | | | | |
| Glycol | | | | | | | | |
| Glycerin | 6 | 4.1 | 4.1 | 4.1 | 4.1 | 4.1 | 4.1 | 4.1 |
| Polyglyceryl-6 Est. | 2.6 | | | | | | | |
| Polyglyceryl-3 | | 20 | 15 | 23 | 8 | 8 | 7.95 | 7.95 |
| Cap. | | | | | | | | |
| Polyglyceryl-2 | 18.5 | 13 | 13 | 24 | 21 | 21.5 | 25 | 26 |
| Cap. | | | | | | | | |
| Cola Lipid C | 4 | 4 | 4 | 4 | 4 | 4 | 4.5 | 4.5 |
| Cola Lipid SAFL | 2 | 2 | 2 | 2 | 2 | 2 | 2 | 2 |
| Phoenamid LMEA | 3 | 3 | 3 | 3 | 3 | 3 | 3 | 3 |
| Stepan Mild L3 | 5 | 7 | 7 | 2 | 7 | 5 | 7 | 7 |
| Nino CAA | 3 | 3 | 3 | 3 | 3 | 3 | 2 | 0 |
| Additives | 4.6 | 4.6 | 4.6 | 4.6 | 3.6 | 4.4 | 3.7 | 3.6 |

The components of each of examples formulation 1-12 were mixed to form hygiene products. The hygiene products are then packaged to form hygiene product pods as described herein. Each product is encapsulated between sheets of film containing a polymer blend of poly(2-ethyl-2-oxazoline) and a blend of PVA polymers with a net hydrolysis of 88%.

### Example 2—Preparation of Exemplary Shelf-Stable Water-Soluble Films

Aqueous polymer solutions were formulated containing: (a) a polymer resin blend with one or more of: polyoxazoline, a polysaccharide, a polypeptide, polyvinyl alcohol, or pyrrolidone, each individually in an amount ranging from about 2 wt % to 80 wt %, (b) one or more plasticizers individually in an amount ranging from about 1 wt % to about 10 wt %, and one or more surfactants in an amount less than about 1 wt %. Each polymer resin, plasticizer, and surfactant was dissolved in cold water mixed with an overhead stirrer for 60 minutes. The solution was kept at 95° C. for 90-180 minutes with continued stirring. Afterwards, the solution was allowed to cool to room temperature with continued stirring. Additives were added before or after dissolving ingredients depending on viscosity. The resulting aqueous mixture is referred to as a polymer slurry. The polymer slurry was cast and left to dry overnight at 74+/−4 Fahrenheit at 50% RH.

### Example 3—Compatibility Testing

Exemplary shelf-stable water-soluble films of those prepared in Example 2 were filled with exemplary hygiene products for compatibility testing. In particular, the hygiene products were encapsulated in a chamber formed between sheets of shelf-stable water-soluble film using a roll coater and a sealing device.

Compatibility of each shelf-stable water-soluble films was tested as follows:

### Dissolution Disintegration of Shelf-Stable Water-Soluble Films

A specimen of each film was cut such that it fit inside a 35 mm slide mount. Each of the samples were locked in a separate 35 mm slide mount. A beaker with 500 ml of distilled water was heated to a temperature to 26 Celsius. The height of the column of water was marked. With the magnetic stirrer engaged, a vortex developed which was one-fifth the height of the column. The depth of the vortex was marked and used as a reference for all future testing. The 35 mm slide mount was clamped in the alligator jaws of a 35 mm slide mount holder such that the long end of the slide mount was parallel to the water surface. The depth adjustment of the clamping device was set so that when dropped, the end of the clamp was 0.6 cm below the surface of the water. One of the short sides of the slide mount was next to the side of the beaker with the other positioned directly over the center of the stirring rod such that the film surface was perpendicular to the flow of the water. In one motion, the secured slide and clamp were dropped into the water and the timer was started. Disintegration occurred when the film breaks. When all visible film is released from the slide mount, the slide was raised out of the water while continuing to monitor the solution for undissolved film fragments. Dissolution occurred when all film fragments were no longer visible and the solution became clear, or the test is stopped at 300 seconds, whichever comes first. Disintegration/dissolution times are reported in Table 3 below.

### Stress Strain Response

The stress strain response was measured by ASTM D882 “Standard Test Method for Tensile Properties of Thin Plastic Sheeting”. The test was conducted on a Texture Analyzer XT.TC plus C with tensile grip attachments. Stress strain response parameters are reported in Table 3 below.

Alternatively, stress strain response of a film can be measured using a Texture Analyzer XT.TC plus C using TA-108S-5i Indexable Film Fixture analyzed under the “Film Burst Test” project. Under this approach, a film is indexed on a fixture and a perpendicular force is applied, consequently obtaining a stress-strain response in the film, which is measured as “Force at Rupture & Distance to Rupture

### Water Content

The resulting water content of each enveloped cosmetic formulation in a shelf-stable water-soluble film was measured by Karl Fischer titration. The film's water content was measured after allowing the film to equilibrate for 72 hours at 74+/− Fahrenheit at 50% RH. Water content of each film is reported in Table 3 below.

TABLE 3
Physical parameters of exemplary films from Example 2

| | Thick- | Disin- | Disso- | Elongation | Tensile | Water |
|---|---|---|---|---|---|---|
| Sample | ness | tegration | lution | at Break | Strength | Content |
| 1 | 3 mil | 17 sec | 19 sec | 620% | 16.70 Mpa | 6.17% |
| 2 | 3 mil | 13 sec | 16 sec | 590% | 17.00 Mpa | 9.87% |

### Pinhole Test

The pinhole test measures the percentage of pouches which leak after thermoforming to encapsulate a liquid cosmetic composition. A sample size of 100 pods is used. The resulting pods are laid upon an absorbing paper and left for 24 hours at 74+/−4 Fahrenheit at 50% RH. Pods demonstrate a pinhole when the liquid composition appear to leak from the thermoformed side of the film, rather than the seal.

### Burst Test

A burst test evaluates the force required to burst a pod. A Texture Analyzer TA.XT plus with compression plate attachments is used to conduct this test. During a burst test, a pod is laid on a stable and strong surface. A perpendicular force is applied, and the force required to burst a pod is measured.

### Exemplary Water Soluble Film Formulation and Cosmetic Formulation Composition Combination

Water soluble films were prepared as described in this Example. Data for an exemplary combination is shown below.

An exemplary soluble film formulation is shown in Table 4 below.

TABLE 4
Water Soluble Film with 3 mil Thickness

| Component | Amount (% weight) |
|---|---|
| Polyvinyl | 40-80% |
| alcohol (>70% | |
| hydrolysis) | |
| Polysaccharide | 1-8% |
| Polyoxazoline | 4-11% |
| Polydextrose | 1-8% |
| Lactic acid | 3-10% |
| Glycol | 3-10% |
| Poly(2-ethyl-2- | 1-8% |
| oxazoline) | |
| Lubricant | 0-5% |
| Sugar alcohol | 1-8% |
| Stearic Acid | 0-5% |
| Ethoxylate | 0-5% |

An exemplary cosmetic composition compatible with the water soluble film described in Table 4 is shown in Table 5, below.

TABLE 5
Exemplary Cosmetic Composition

| Component | Amount (% weight) |
|---|---|
| Cocamidopropyl Betaine | 1-10% |
| Glucotain Plus | 10-15% |
| Propylene Glycol | 5-10% |
| Glycerin | 1-5% |
| Polyglyceryl-2 Caprate | 0-30% |
| Secondary surfactant (sulfate-free) | 10-15% |
| Glycerides | 1-10% |
| Emollient | 1-10% |
| Conditioning agent | 0-10% |
| Fragrance | 0-5% |

The compatibility data for the water soluble film described in Table 4 and the cosmetic composition described in Table 5 are shown below in Table 6.

TABLE 6
Exemplary compatibility data for a water
soluble film and a cosmetic composition

| Analysis Parameter | Day 14 | Day 28 | Day 42 |
|---|---|---|---|
| Disintegration (sec) | 18 | 20 | 23 |
| Dissolution (sec) | 24 | 24 | 27 |
| Cosmetic Formulation | 12.47% | 12.90% | 13.37% |
| Water Content (%) | | | |
| Force at Burst (g) | 1849 | 1803 | 1786 |
| Distance to Burst (mm) | 14.38 | 15.02 | 15.87 |

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1. A shelf-stable water-soluble film comprising:
(a) a hydrophilic polymer blend with a polymeric resin weight average molecular weight of from about 20,000 g/mol to about 80,000 g/mol, wherein the hydrophilic polymer blend comprises:
(i) a polyoxazoline polymer,
(ii) a polysaccharide, or
(iii) a polypeptide; and
(b) water content of at least about 1 wt %, with respect to a total weight of the shelf-stable water-soluble film; wherein
the shelf-stable water-soluble film at a thickness of about 3 mil when contacted with water at 26° C. has a disintegration time of less than about 30 seconds, and wherein
the shelf-stable water-soluble film exhibits stability and lack of degradation for at least about 14 days when stored at 38° C. and 80% relative humidity.

2. A shelf-stable water-soluble film comprising:
(c) a hydrophilic polymer blend with a polymeric resin weight average molecular weight of from about 20,000 g/mol to about 80,000 g/mol, wherein the hydrophilic polymer blend comprises:
(i) a polyoxazoline polymer,
(ii) a polysaccharide, or
(iii) a polypeptide; and
(d) water content of at least about 1 wt %, with respect to a total weight of the shelf-stable water-soluble film; wherein
the shelf-stable water-soluble film at a thickness of about 3 mil when contacted with water at 26° C. has a disintegration time of less than about 60 seconds, and wherein
the shelf-stable water-soluble film has a tensile strength of from about 10 Mpa to about 30 Mpa, as determined by a universal tensile tester.

3. The shelf-stable water-soluble film of claim 1, wherein the shelf-stable water-soluble film at a thickness of about 3 mil has a shorter disintegration time when contacted with water at 26° C., relative to a disintegration time under the same conditions of an otherwise comparable film that does not comprise the polyoxazoline polymer, the polysaccharide, or the polypeptide.

4. The shelf-stable water-soluble film of claim 1, wherein the hydrophilic polymer blend further comprises a polyvinyl alcohol polymer.

5. The shelf-stable water-soluble film of claim 4, wherein the polyvinyl alcohol polymer is present in a total amount of from about 50% to about 80% by weight, with respect to a total weight of the shelf-stable water-soluble film.

6. The shelf-stable water-soluble film of claim 4, wherein the polyvinyl alcohol polymer has a net degree of hydrolysis of from about 75% to about 90%.

7. The shelf-stable water-soluble film of claim 1, wherein the water content is from about 1 wt % to about 10 wt %, with respect to the total weight of the shelf-stable water-soluble film.

8. The shelf-stable water-soluble film of claim 1, wherein the hydrophilic polymer blend comprises the polypeptide.

9. The shelf-stable water-soluble film of claim 8, wherein the polypeptide is casein, zein, wheat gluten, whey protein, soy protein, collagen or gelatin.

10. The shelf-stable water-soluble film of claim 1, wherein the hydrophilic polymer blend comprises the polysaccharide.

11. The shelf-stable water-soluble film of claim 10, wherein the polysaccharide is selected from the group consisting of: hydroxypropyl methyl cellulose, carboxymethyl cellulose, methyl ether cellulose, microcrystalline cellulose, chitosan, pullulan, methoxyl pectin, curdlan, ceral beta glucan, gellan, konjac glucomannan, polydextrose, carrageenan, alginic acid, laminarin, mannan, mannitol, porphyrin, xylan, and a salt of any of these.

12. The shelf-stable water-soluble film of claim 1, wherein the hydrophilic polymer blend comprises the polyoxazoline polymer.

13. The shelf-stable water-soluble film of claim 12, wherein the polyoxazoline polymer is poly(2-ethyl-2-oxazoline).

14. The shelf-stable water-soluble film of claim 13, wherein the poly(2-ethyl-2-oxazoline) is present in an amount of from about 1 wt % to about 25 wt %, with respect to a total weight of the shelf-stable water-soluble film.

15.-19. (canceled)

20. The shelf-stable water-soluble film of claim 2, wherein the shelf-stable water-soluble film has an elongation at break of at least about 500%.

21.-37. (canceled)

38. A hygiene product pod comprising:
(e) a water-soluble envelope, wherein the water-soluble envelope comprises a hydrophilic polymer blend that comprises:
(i) a polyoxazoline polymer or a polypeptide; and
(ii) a water-containing liquid hygiene product sealed in the water-soluble envelope, wherein the water-containing liquid hygiene product does not phase separate within the water-soluble envelope; and
wherein when a group of about 100 hygiene product pods are stored for at least 24 hours at 23° C. and 50% relative humidity, less than 5 of the group of about 100 hygiene product pods degrade, as determined by leakage of the water-containing liquid hygiene product.

39.-51. (canceled)

52. A hygiene product pod comprising:
(a) a water-soluble envelope, wherein the water-soluble envelope comprises:
(i) a hydrophilic polymer blend with a polymeric resin weight average molecular weight of from about 20,000 g/mol to about 80,000 g/mol, wherein the hydrophilic polymer blend comprises:
1) poly(2-ethyl-2-oxazoline) at a molecular weight of at least 20,000 g/mol, and
2) a blend of at least two polyvinyl alcohol polymers that have a net degree of hydrolysis of from about 75% to about 90%,
(ii) water content of from about 1 wt % to about 10 wt %, with respect to a total weight of the water-soluble envelope, and
(iii) less than about 5 wt % of a polyhydric alcohol with a molecular weight less than about 200 g/mol, with respect to a total weight of the shelf-stable water-soluble film;
(b) a water-containing liquid hygiene product sealed in the water-soluble envelope, wherein the water-containing liquid hygiene product comprises, based on a total weight of the water-containing liquid hygiene product:
(i) a non-aqueous carrier comprising one or more polyol solvents in an amount ranging from about 10 wt % to about 30 wt %,
(ii) water content of less than about 25 wt %,
wherein when a group of about 100 hygiene product pods are stored for at least 24 hours at 23° C. and 50% relative humidity, less than 5 of the group of about 100 hygiene product pods degrade, as determined by leakage of the water-containing liquid hygiene product.

53. The hygiene product pod of claim 52, wherein the water-containing liquid hygiene product further comprises a polyglyceryl fatty acid ester (PGE) chassis in an amount ranging up to 50 wt %, based on a total weight of the water-containing liquid hygiene product.

54. A method of making a shelf-stable water-soluble hygiene product pod, comprising:
(a) dissolving a hydrophilic polymer blend comprising a polyoxazoline polymer or a polypeptide in water;
(b) heating the hydrophilic polymer blend, thereby generating a polymer blend film; and
(c) encapsulating a liquid hygiene product in the polymer film, thereby generating the shelf-stable water-soluble hygiene product pod.
